(19) United States
(12) Patent Application Publication
(10) Pub. No.: US 2020/0345648 A1
(43) Pub. Date: Nov. 5, 2020

(54) METHODS AND COMPOSITIONS FOR TREATING CANCER USING EXOSOMES-ASSOCIATED GENE EDITING

(71) Applicant: Board of Regents, The University of Texas System, Austin, TX (US)

(72) Inventors: Raghu Kalluri, Houston, TX (US); Valerie LeBleu, Houston, TX (US); Fei Xiao, Houston, TX (US)

(21) Appl. No.: 16/772,759

(22) PCT Filed: Dec. 14, 2018
(86) PCT No.: PCT/US2018/065642

§ 371 Date: Jun. 12, 2020

Related U.S. Application Data
(60) Provisional application No. 62/599,340, filed on Dec. 15, 2017.

(51) Int. Cl. A61K 9/50; C12N 15/88; C12N 15/11; C12N 9/22; A61K 9/00; A61K 45/06; A61K 38/46; A61K 31/7088; A61P 35/00

(52) U.S. Cl. CPC A61K 9/5068; C12N 15/88; C12N 15/11; C12N 9/22; A61K 9/0029; C12N 2800/80; A61K 45/06; A61K 38/465; A61K 31/7088; A61P 35/00; C12N 2310/20; A61K 9/0019

(57) ABSTRACT

Provided herein are compositions comprising exosomes comprising CD47 on their surface, and further comprising a CRISPR system. Further provided are methods of using the exosomes for gene editing and the treatment of cancer by gene editing.

### REFERENCE TO RELATED APPLICATIONS

The present application claims the priority benefit of U.S. provisional application No. 62/599,340, filed Dec. 15, 2017, the entire contents of which is incorporated herein by reference.

### BACKGROUND

### 1. Field

The present invention relates generally to the fields of medicine and oncology. More particularly, it concerns the use of exosomes for the in vivo delivery of nuclease complexes for gene editing.

### 2. Description of Related Art

Gene editing is a technology that allows for the modification of target genes within living cells. Recently, harnessing the bacterial immune system of CRISPR to perform on demand gene editing revolutionized the way scientists approach genomic editing. The Cas9 protein of the CRISPR system, which is an RNA guided DNA endonuclease, can be engineered to target new sites with relative ease by altering its guide RNA sequence. This discovery has made sequence specific gene editing functionally effective. The current CRISPR/Cas9 technology offers reliable methods to edit genes in cultured cells in vitro; however, new methods of targeting specific cells in different organs in vivo are needed.

### SUMMARY

As such, exosomes engineered to carry CRISPR-Cas9 to different organs and tumors with high efficiency are provided, thereby enabling therapeutic gene editing to control cancer and other genetic diseases. In one embodiment, compositions comprising exosomes are provided, wherein the exosomes comprise CD47 on their surface and wherein the exosomes comprise a CRISPR system. In some aspects, the CRISPR system comprises an endonuclease and a guide RNA (gRNA). In some aspects, the endonuclease is a Cas endonuclease. In some aspects, the endonuclease is a Cas9 endonuclease. In other aspects, the endonuclease is a Cpf1 endonuclease. In some aspects, the guide RNA is a single gRNA. In some aspects, the single gRNA is a CRISPR-RNA (crRNA). In some aspects, the single gRNA comprises a fusion of a crRNA and a trans-activating CRISPR RNA (tracrRNA). In some aspects, the guide RNA comprises a crRNA and a tracrRNA. In some aspects, the endonuclease and the gRNA are encoded on a single nucleic acid molecule within the exosomes. In some aspects, the endonuclease and the gRNA are encoded on separate nucleic acid molecules within the exosomes.

In some aspects, the CRISPR system targets a disease-causing mutation. In some aspects, the disease-causing mutation is a cancer-causing mutation. In some aspects, the cancer-causing mutation is an activating mutation in an oncogene. In some aspects, the cancer-causing mutation is an inhibitory mutation in a tumor suppressor gene. In some aspects, the CRISPR system targets an undruggable gene. In some aspects, the cancer-causing mutation is Kras^{G12D}. In some aspects, at least 2%, 5%, 10%, 15%, 20%, 25%, 30%, 35%, 40%, 45%, 50%, 55%, 60%, 65%, 70%, 75%, 80%, 85%, 90%, 95%, or 98% (or any value derivable therein) of the exosomes comprise an endonuclease and a gRNA.

In one embodiment, pharmaceutical compositions comprising exosomes and a pharmaceutically acceptable excipient are provided, wherein the exosomes comprise CD47 on their surface and wherein the exosomes comprise a CRISPR system. In some aspects, the CRISPR system comprises an endonuclease and a guide RNA (gRNA). In some aspects, the endonuclease is a Cas endonuclease. In some aspects, the endonuclease is a Cas9 endonuclease. In other aspects, the endonuclease is a Cpf1 endonuclease. In some aspects, the guide RNA is a single gRNA. In some aspects, the single gRNA is a CRISPR-RNA (crRNA). In some aspects, the single gRNA comprises a fusion of a crRNA and a trans-activating CRISPR RNA (tracrRNA). In some aspects, the guide RNA comprises a crRNA and a tracrRNA. In some aspects, the endonuclease and the gRNA are encoded on a single nucleic acid molecule within the exosomes. In some aspects, the endonuclease and the gRNA are encoded on separate nucleic acid molecules within the exosomes. In some aspects, the CRISPR system targets a disease-causing mutation. In some aspects, the disease-causing mutation is a cancer-causing mutation. In some aspects, the cancer-causing mutation is an activating mutation in an oncogene. In some aspects, the cancer-causing mutation is an inhibitory mutation in a tumor suppressor gene. In some aspects, the CRISPR system targets an undruggable gene. In some aspects, the cancer-causing mutation is Kras^{G12D}. In some aspects, at least 2%, 5%, 10%, 15%, 20%, 25%, 30%, 35%, 40%, 45%, 50%, 55%, 60%, 65%, 70%, 75%, 80%, 85%, 90%, 95%, or 98% (or any value derivable therein) of the exosomes comprise an endonuclease and a gRNA. In some aspects, the composition is formulated for parenteral administration. In some aspects, the composition is formulated for intravenous, intramuscular, sub-cutaneous, or intraperitoneal injection. In further aspects, the composition further comprises an antimicrobial agent. In some aspects, the antimicrobial agent is benzalkonium chloride, benzethonium chloride, benzyl alcohol, bronopol, centrimide, cetylpyridinium chloride, chlorhexidine, chlorobutanol, chlorocresol, chloroxylenol, cresol, ethyl alcohol, glycerin, exetidine, imidurea, phenol, phenoxyethanol, phenylethl alcohol, phenlymercuric nitrate, propylene glycol, or thimerosal.

In one embodiment, methods of treating a disease in a patient in need thereof are provided, said methods comprising administering to the patient a composition comprising a pharmaceutical composition comprising exosomes and a pharmaceutically acceptable excipient, wherein the exosomes comprise CD47 on their surface and wherein the exosomes comprise a CRISPR system, thereby treating the disease in the patient. In some aspects, administration causes gene editing in the diseased cells in the patient. In some aspects, the disease is a cancer. In some aspects, the cancer is pancreatic ductal adenocarcinoma. In some aspects, the administration is systemic administration. In some aspects, the systemic administration is intravenous or intraarterial administration. In some aspects, the method further comprises administering at least a second therapy to the patient. In some aspects, the second therapy comprises a surgical therapy, chemotherapy, radiation therapy, cryotherapy, hormonal therapy, or immunotherapy. In some aspects, the patient is a human. In some aspects, the exosomes are autologous to the patient. In some aspects, administration of the pharmaceutical composition provides superior therapeutic benefit relative to administration of an exosomes-free CRISPR system. In some aspects, the pharmaceutical composition is administered to the patient only one. In some aspects, the pharmaceutical composition is administered to the patient more than once. In some aspects, the pharmaceutical composition is administered to the patient a finite number of times. In some aspects, the pharmaceutical composition is administered to the patient continuously. In some aspects, the pharmaceutical composition is administered to the patient at least 2, 3, 4, 5, 6, 7, 8, 9, 10, 11, 21, 13, 14, 15, 20, 25, 30, 35, 40, 45, or 50 (or any value derivable therein) times.

In one embodiment, compositions comprising exosomes for use in the treatment of a disease in a patient are provided, wherein the exosomes comprise CD47 on their surface and wherein the exosomes comprises a CRISPR system. In some aspects, the CRISPR system comprises an endonuclease and a guide RNA (gRNA). In some aspects, the endonuclease is a Cas endonuclease. In some aspects, the endonuclease is a Cas9 endonuclease. In other aspects, the endonuclease is a Cpf1 endonuclease. In some aspects, the guide RNA is a single gRNA. In some aspects, the single gRNA is a CRISPR-RNA (crRNA). In some aspects, the single gRNA comprises a fusion of a crRNA and a trans-activating CRISPR RNA (tracrRNA). In some aspects, the guide RNA comprises a crRNA and a tracrRNA. In some aspects, the endonuclease and the gRNA are encoded on a single nucleic acid molecule within the exosomes. In some aspects, the endonuclease and the gRNA are encoded on separate nucleic acid molecules within the exosomes. In some aspects, the CRISPR system targets a disease-causing mutation. In some aspects, the disease-causing mutation is a cancer-causing mutation. In some aspects, the cancer-causing mutation is an activating mutation in an oncogene. In some aspects, the cancer-causing mutation is an inhibitory mutation in a tumor suppressor gene. In some aspects, the CRISPR system targets an undruggable gene. In some aspects, wherein the cancer-causing mutation is Kras^{G12D}. In some aspects, at least 2%, 5%, 10%, 15%, 20%, 25%, 30%, 35%, 40%, 45%, 50%, 55%, 60%, 65%, 70%, 75%, 80%, 85%, 90%, 95%, or 98% (or any value derivable therein) of the exosomes comprise an endonuclease and a gRNA. In some aspects, administration causes gene editing in the diseased cells in the patient. In some aspects, the disease is a cancer. In some aspects, the cancer is pancreatic ductal adenocarcinoma. In some aspects, the composition is formulated for parenteral administration. In some aspects, the composition is formulated for intravenous, intramuscular, sub-cutaneous, or intraperitoneal injection. In further aspects, the composition further comprises an antimicrobial agent. In some aspects, the antimicrobial agent is benzalkonium chloride, benzethonium chloride, benzyl alcohol, bronopol, centrimide, cetylpyridinium chloride, chlorhexidine, chlorobutanol, chlorocresol, chloroxylenol, cresol, ethyl alcohol, glycerin, exetidine, imidurea, phenol, phenoxyethanol, phenylethl alcohol, phenlymercuric nitrate, propylene glycol, or thimerosal. In a further aspect, the composition comprises at least a second therapy. In some aspects, the second therapy comprises a surgical therapy, chemotherapy, radiation therapy, cryotherapy, hormonal therapy, or immunotherapy. In some aspects, the patient is a human. In some aspects, the exosomes are autologous to the patient.

In one embodiment, uses of exosomes in the manufacture of a medicament for the treatment of a disease are provided, wherein the exosomes comprise CD47 on their surface and wherein the exosomes comprise a CRISPR system. In some aspects, the CRISPR system comprises an endonuclease and a guide RNA (gRNA). In some aspects, the endonuclease is a Cas endonuclease. In some aspects, the endonuclease is a Cas9 endonuclease. In other aspects, the endonuclease is a Cpf1 endonuclease. In some aspects, the guide RNA is a single gRNA. In some aspects, the single gRNA is a CRISPR-RNA (crRNA). In some aspects, the single gRNA comprises a fusion of a crRNA and a trans-activating CRISPR RNA (tracrRNA). In some aspects, the guide RNA comprises a crRNA and a tracrRNA. In some aspects, the endonuclease and the gRNA are encoded on a single nucleic acid molecule within the exosomes. In some aspects, the endonuclease and the gRNA are encoded on separate nucleic acid molecules within the exosomes. In some aspects, the CRISPR system targets a disease-causing mutation. In some aspects, the disease-causing mutation is a cancer-causing mutation. In some aspects, the cancer-causing mutation is an activating mutation in an oncogene. In some aspects, the cancer-causing mutation is an inhibitory mutation in a tumor suppressor gene. In some aspects, the CRISPR system targets an undruggable gene. In some aspects, the cancer-causing mutation is Kras^{G12D}. In some aspects, at least 2%, 5%, 10%, 15%, 20%, 25%, 30%, 35%, 40%, 45%, 50%, 55%, 60%, 65%, 70%, 75%, 80%, 85%, 90%, 95%, or 98% (or any value derivable therein) of the exosomes comprise an endonuclease and a gRNA. In some aspects, the disease is a cancer. In some aspects, the cancer is pancreatic ductal adenocarcinoma. In some aspects, the medicament is formulated for parenteral administration. In some aspects, the medicament is formulated for intravenous, intramuscular, sub-cutaneous, or intraperitoneal injection. In some aspects, the medicament comprises an antimicrobial agent. In some aspects, the antimicrobial agent is benzalkonium chloride, benzethonium chloride, benzyl alcohol, bronopol, centrimide, cetylpyridinium chloride, chlorhexidine, chlorobutanol, chlorocresol, chloroxylenol, cresol, ethyl alcohol, glycerin, exetidine, imidurea, phenol, phenoxyethanol, phenylethl alcohol, phenlymercuric nitrate, propylene glycol, or thimerosal.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects. Other objects, features and advantages of the present invention will become apparent from the following detailed description. It should be understood, however, that the detailed description and the specific examples, while indicating certain embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1a-h: HEK293T cells were transfected with CRISPR-Cas9 vector control and CRISPR-Cas9-sgRab27a-2 using lipofectamine for 72 h and then selected with 1 μg/ml puromycin for 10 days to obtain stable HEK293T CRISPR-Cas9 vector control and CRISPR-Cas9-sgRab27a-2 cells. The stables cells were cultured with 1 μg/ml puromycin containing selection medium. (FIG. 1a) DNA and RNA were extracted from the abovementioned cells, and Cas9 levels were determined using quantitative real-time PCR (qPCR). (FIG. 1b) Exosomes were collected from HEK293T blank cells, as well as stable HEK293T CRISPR-Cas9 vector control and CRISPR-Cas9-sgRab27a-2 cells, followed by Nanosight validation. (FIG. 1c) Exosomal DNA and RNA were extracted, and qPCR was performed to detect Cas9 levels in exosomes, as well as sgRNA against Rab27a-2. (FIG. 1d) Cas9 protein levels were assessed in both cells and exosomes by Western blot, using either anti-Flag antibody or Cas9 antibody, with Vinculin or CD9 as controls, respectively. (FIG. 1e) and (FIG. 1f) T7/SURVEYOR assay was used to determine DNA editing in both cells (FIG. 1e) and exosomes (FIG. 1f). (FIG. 1g) and (FIG. 1h) 3E10 exosomes collected from HEK293T blank cells, HEK293T CRISPR-Cas9 vector control and CRISPR-Cas9-sgRab27a-2 stable cells were treated into BxPC-3 every 24 h, + treated once, ++ treated twice. DNA and RNA were extracted from the recipient cells. Cas9 levels were detected in both DNA (g) and mRNA (h) level. The bars at each time point represent, from left to right, “Blank control,” “CRISPR-Cas9 Vector control,” and “CRISPR-Cas9-sgRab27a-2.”

FIGS. 2a-c: 3E10 exosomes collected from HEK293T blank cells, HEK293T CRISPR-Cas9 vector control and CRISPR-Cas9-sgRab27a-2 stable cells were treated into BxPC-3 every 24 h twice. DNA and RNA were extracted from the recipient cells. (FIG. 2a) and (FIG. 2c) sgRNA against Rab27a-2 was detected by PCR in both DNA (FIG. 2a) and mRNA (FIG. 2c) level. (FIG. 2b) T7/SURVEYOR assay was used to determine DNA editing in the recipient BxPC-3 cells.

FIGS. 3a-d: Exosomes were collected from BJ cells. (FIG. 3a) Nanosight was used to validate the exosomes. (FIG. 3b) Exosome markers CD9, CD81, Flotillin and TSG101 were detected by Western blot to further confirm the exosomes. (FIG. 3c) 1E10 BJ exosomes were electroporated with 15 ug CRISPR-Cas9-GFP plasmid, and then treated with or without DNase. Exosomal DNA was extracted and Cas9 level was evaluated by qPCR. Copy number was further calculated by absolute qPCR with CRISPR-Cas9-GFP plasmid as a standard. (FIG. 3d) The electroporated exosomes with DNase were treated into BJ cells for 24 h. Cas9 levels were detected in both DNA and mRNA level.

FIGS. 4a-b: HEK293T cells were transfected using packaging plasmids together with CRISPR-Cas9 Rab27b-1/2 or empty control plasmids by lipofectamine 2000. The medium containing lentivirus was harvested and then transduced into BxPC-3 cells. The transduced cells were further selected with 0.4 μg/mL puromycin, and single clones of BxPC-3/CRISPR-Cas9-sgRab27b cells were picked up, expanded and validated by both Western blot and T7/SURVEYOR assay. (FIG. 4a) Rab27b and Rab27a protein levels were evaluated in all the single clones, with (3-actin as a loading control. Representative Western blot results were shown in (FIG. 4a). (FIG. 4b) T7/SURVEYOR assay was also used to further validate all the clones. Representative Western blot results were shown in (FIG. 4b). BxPC-3/CRIPSR-Cas9-sgRab27b-1 clone 3 (C3) and BxPC-3/CRISPR-Cas9-sgRab27b-2 clone 6 (C6) were used for further experiments.

FIGS. 5a-f: BxPC-3/CRISPR-Cas9 vector control stable cells and single clones BxPC-3/CRISPR-Cas9-sgRab27b-1 C3, BxPC-3/CRISPR-Cas9-sgRab27b-2 C6 were cultured with 0.4 μg/ml puromycin containing selection medium. (FIG. 5a) DNA and RNA were extracted from the abovementioned cells, and Cas9 levels were determined using qPCR. (FIG. 5b) Exosomes were collected from the abovementioned cells, followed by Nanosight validation. Secreted exosome numbers were analyzed and compared by Nanosight. (FIG. 5c) Exosomal DNA and RNA were extracted, and qPCR was performed to detect Cas9 levels in exosomes, as well as sgRNA against Rab27b-1/2. (FIG. 5d) Cas9 and Rab27b protein levels were assessed in both cells and exosomes by Western blot, with b-actin or CD9 as controls, respectively. (FIG. 5e) and (FIG. 5f) T7/SURVEYOR assay was used to determine DNA editing in both cells (FIG. 5e) and exosomes (FIG. 5f) using two different primer sets.

FIGS. 6a-b: (FIG. 6a) Exosomes collected from BxPC-3/CRISPR-Cas9 vector control stable cells and single clones BxPC-3/CRISPR-Cas9-sgRab27b-1 C3, BxPC-3/CRISPR-Cas9-sgRab27b-2 C6 were lysed and protein content was further detected by BCA kit according to the manufacturer's instructions. (FIG. 6b) 100 μL of BxPC-3 blank, BxPC-3/CRISPR-Cas9 empty control, BxPC-3/CRISPR-Cas9-sgRab27b-1 C3 and BxPC-3/CRISPR-Cas9-sgRab27b-2 C6 cells were seeded in 96-well plates at the concentration of 1E5 cells/ml. Cell proliferation was evaluated using MTT assay at different time points. The bars at each time point represent, from left to right, “Blank control,” “CRISPR-Cas9 Vector control,” “CRISPR-Cas9-sgRab27b-1-C3,” and “CRISPR-Cas9-sgRab27b-2-C6.”

FIGS. 7a-g: (FIG. 7a) To generate in vitro transcribed sgRab27b, sgRab27b-1/2 was first amplified by PCR, and then the PCR products were purified using the Qiagen® PCR purification kit. The purified PCR products of sgRab27-1/2 were in vitro transcribed using the MEGAshortscript™ kit according to the manufacturer's instructions. The RNA quality was further evaluated by 8M urea polyacrylamide gel. (FIG. 7b) To generate In vitro transcribed Cas9, Cas9 was amplified by PCR, with the PCR products further purified using the Qiagen® PCR purification kit. Purified Cas9 PCR products were in vitro transcribed using the mMESSAGE mMACHINE® T7 Ultra Kit. Formaldehyde gels were used to detect Cas9 RNA quality. (FIGS. 7c-e) HEK293T/CRISPR-Cas9 vector control cells were treated with 1 μg IVT-sgRab27b RNA using lipofectamine 2000 (FIG. 7c), Exo-Fect/exosome transfection reagent (FIG. 7d) or electroporated exosomes (FIG. 7e) for 72 h. DNA was extracted, and T7/SURVEYOR assay was performed to check gene editing. HEK293T cells (FIG. 7f) and BxPC-3 cells (FIG. 7g) were transfected with Cas9 mRNA using lipofectamine 2000, Exo-Fect/exosome transfection reagent, or treated with 1E9 MSC exosomes electroporated with Cas9 mRNA for 48 h. Western blot was performed to detect Cas9 protein level.

FIGS. 8a-c: RNA was extracted from HEK293T/CRISPRCas9 vector control and BxPC-3/CRISPR-Cas9 vector control cells. Relative Cas9 expression level (FIG. 8a) and 1/Ct value (FIG. 8b) were determined by qPCR. (FIG. 8c) 1 μg Cas9 RNA was used for reverse transcription together with RNAs from HEK293T/CRISPRCas9 vector control and BxPC-3/CRISPR-Cas9 vector control cells. qPCR was performed to detect 1/Ct value.

FIGS. 9a-g: HEK293T cells were treated with 10 μg plasmids (CRISPR-Cas9-lenti-V2 vector control, CRISPR-Cas9-lenti-V2-sgRab27b-1, CRISPR-Cas9-GFP vector control) using Exo-Fect/exosome transfection reagent every 24 h for 4 times (day 1, 2, 3, 4). Cells were collected on day 5. DNA, RNA and protein were extracted. (FIG. 9a) Pictures taken on day 5 were shown to represent the transfection efficiency of Exo-Fect/exosome transfection reagent by using CRISPR-Cas9-GFP vector control plasmid as a control. (FIG. 9b) Relative Cas9 expression level and 1/Ct value were determined by qPCR. (FIG. 9c) Western blot was used to evaluate Cas9 protein level. (FIG. 9d) T7/SURVEYOR assay was performed to check gene editing in HEK293T cells after treated with CRISPR-Cas9-lenti-V2-sgRab27b-1 plasmid. Same experiment was performed in BxPC-3 cells. BxPC-3 cells were treated with 10 μg plasmids (CRISPRCas9-lenti-V2 vector control, CRISPR-Cas9-lenti-V2-sgRab27b-1) using Exo-Fect/exosome transfection reagent every 24 h for 4 times (day 1, 2, 3, 4). Cells were collected on day 5. (FIG. 9e) Relative Cas9 expression level was determined by qPCR. (FIG. 9f) Western blot was used to evaluate Cas9 protein level. (FIG. 9g) T7/SURVEYOR assay was performed to check gene editing in BxPC-3 cells.

FIGS. 10a-h: KPC689 cells were transfected with 5 μg plasmids (CRISPR-Cas9-sgmKras^{G12D} with lenti-V2, GFP, puromycin backbone, and the vector controls) by lipofectamine 2000 for 48 h. DNA, RNA and protein were extracted. (FIG. 10a) Pictures were taken after transfection for 48 h to represent the transfection efficiency of lipofectamine by using CRISPR-Cas9-GFP vector control plasmid as a control. Relative Cas9 expression level (FIG. 10b) and mKras^{G12D} level (FIG. 10c) were determined by qPCR. (FIG. 10d) T7/SURVEYOR assay was performed to check gene editing in KPC689 cells after transfection by lipofectamine KPC689 cells were treated with 10 μg plasmids (CRISPR-Cas9-sgmKras^{G12D} with GFP backbone, and its vector control) using Exo-Fect/exosome transfection reagent every 24 h for 3 times (day 1, 2, 3). Cells were collected on day 4. DNA, RNA and protein were extracted. (FIG. 10e) Pictures taken on day 5 were shown to represent the transfection efficiency of Exo-Fect/exosome transfection reagent. Relative Cas9 expression level (FIG. 10f) and mKras^{G12D} level (FIG. 10g) were determined by qPCR. (FIG. 10h) T7/SURVEYOUR assay was performed to check gene editing in KPC689 cells after treated with CRISPR-Cas9-GFP-mKras^{G12D} plasmids.

FIGS. 11a-f: (FIG. 11a) and (FIG. 11b) HEK293T cells were transfected using packaging plasmids together with CRISPR-Cas9 doxycycline inducible plasmid by lipofectamine 2000. The medium containing lentivirus was harvested and then transduced into Panc1 cells. The transduced cells were further selected with 1 μg/ml puromycin. The Panc1 inducible Cas9 stable cells were maintained using 1 μg/ml doxycycline. Exosomes were collected from Panc1 inducible cells treated with or without doxycycline. Western blot was used to check Cas9 protein level in cells (FIG. 11a) and exosomes (FIG. 11b). (FIG. 11c) The Panc1 inducible cells were treated with 2 μg IVT-sgRNA against hKras^{G12D}, 1 μg hKras^{G12D} plasmid by lipofectamine, Fugene or Exo-Fect for 72 h. T7/SURVEYOR assay was performed to check gene editing in Panc1 inducible cells. (FIG. 11d) Panc1 Cas9 stable cells were established using lentivirus based method. Cas9 protein level was determined by Western blot. (FIG. 11e) Panc1 cells were treated with CRISPR-Cas9-sghKras^{G12D} with lenti-V2, GFP, puromycin backbones using lipofectamine, Exo-Fect or electroporated exosomes. Panc1 Cas9 stable cells were treated with sghKras^{G12D} plasmids using lipofectamine, Exo-Fect or electroporated exosomes. T7/SURVEYOR assay was performed to check gene editing in Panc1 cells and Panc1 Cas9 stable cells. (FIG. 11f) Panc1 sghKras^{G12D} T1 stable cells were established using lentivirus based method. The Panc1 sghKras^{G12D} T1 stable cells were transfected with 10 μg or 20 μg Cas9 plasmids with either GFP or puromycin backbone for 24 h. T7/SURVEYOR assay was performed to check gene editing in Panc1 sghKras^{G12D} T1 stable cells.

FIGS. 12a-b: KPC689 cells were implanted subcutaneously into the back of the mice. The mice were divided into 4 groups, with 1 or 2 mice per group. Group 1: treated with 1E9 exosomes and 10 μl Exo-Fect (n=1, K504); group 2: treated with 10 μg Cas9-GFP-sgmKras^{G12D}-mK1 plasmid (n=1, K509); group 3: treated with 1E9 exosomes, 10 μg Cas9-GFP-vector control plasmid and 10 μl Exo-Fect (n=2, #1: K501, #2: K510. K510 enrolled 3 days later, compared with all the other mice); group 4: treated with 1E9 exosomes, 10 μg Cas9-GFP-sgmKras^{G12D}-mK1 plasmid and 10 μl Exo-Fect (n=2, #1: K502, #2: K505). Mice in each group were injected intravenously (I.V.) and intratumorally (I.T.) every day for two weeks. (FIG. 12a) Tumor length (a, mm) and width (b, mm) as well as body weight (FIG. 12b) were measured. Tumor volume (FIG. 12a) was calculated as V (mm³)=0.52*a*b\hat{ }2.

### DETAILED DESCRIPTION

Provided herein are exosomes (e.g., iExosomes^{CRISPR/Cas9}) having an incorporated CRISPR/Cas9 system using different guide RNA molecules with the ability to target cancer cells and induce a gene-editing program to alter the genome of the cancer cells. Gene-editing assays have been used to show that gene editing occurred efficiently in the exosomes themselves, offering a rapid validation method for efficiency and subsequent use of the iExosomes^{CRISPR/Cas9} to target cancer cells with mutations, such as Kras^{G12D}, to edit the mutated gene out and replace it with a wild-type KRAS gene or remove the dominant mutant gene and allow for the normal gene to take over the function. Using iExosomes^{CRISPR/Cas9} any gene that is part of the genomic DNA of cancer cells and tumors in general that are contributing to the initiation, progression, and/or metastasis can be edited to provide therapeutic benefit or change the biology of cancer cells and the tumors. This technology overcomes the lack of in vivo application of CRISPR/Cas9 technology currently for cancer-associated gene editing with therapeutic benefit. Using exosomes with CD47 on the surface, iExosomes^{CRISPR/Cas9} can be successfully delivered to tumors for therapeutic benefit.

### I. LIPID-BASED NANOPARTICLES

In some embodiments, a lipid-based nanoparticle is a liposomes, an exosomes, lipid preparations, or another lipid-based nanoparticle, such as a lipid-based vesicle (e.g., a DOTAP:cholesterol vesicle). Lipid-based nanoparticles may be positively charged, negatively charged or neutral.

A. Liposomes

A “liposome” is a generic term encompassing a variety of single and multilamellar lipid vehicles formed by the generation of enclosed lipid bilayers or aggregates. Liposomes may be characterized as having vesicular structures with a bilayer membrane, generally comprising a phospholipid, and an inner medium that generally comprises an aqueous composition. Liposomes provided herein include unilamellar liposomes, multilamellar liposomes, and multivesicular liposomes. Liposomes provided herein may be positively charged, negatively charged, or neutrally charged. In certain embodiments, the liposomes are neutral in charge.

A multilamellar liposome has multiple lipid layers separated by aqueous medium. Such liposomes form spontaneously when lipids comprising phospholipids are suspended in an excess of aqueous solution. The lipid components undergo self-rearrangement before the formation of closed structures and entrap water and dissolved solutes between the lipid bilayers. Lipophilic molecules or molecules with lipophilic regions may also dissolve in or associate with the lipid bilayer.

In specific aspects, a polypeptide, a nucleic acid, or a small molecule drug may be, for example, encapsulated in the aqueous interior of a liposome, interspersed within the lipid bilayer of a liposome, attached to a liposome via a linking molecule that is associated with both the liposome and the polypeptide/nucleic acid, entrapped in a liposome, complexed with a liposome, or the like.

A liposome used according to the present embodiments can be made by different methods, as would be known to one of ordinary skill in the art. For example, a phospholipid, such as for example the neutral phospholipid dioleoylphosphatidylcholine (DOPC), is dissolved in tert-butanol. The lipid(s) is then mixed with a polypeptide, nucleic acid, and/or other component(s). Tween 20 is added to the lipid mixture such that Tween 20 is about 5% of the composition's weight. Excess tert-butanol is added to this mixture such that the volume of tert-butanol is at least 95%. The mixture is vortexed, frozen in a dry ice/acetone bath and lyophilized overnight. The lyophilized preparation is stored at −20° C. and can be used up to three months. When required the lyophilized liposomes are reconstituted in 0.9% saline.

Alternatively, a liposome can be prepared by mixing lipids in a solvent in a container, e.g., a glass, pear-shaped flask. The container should have a volume ten-times greater than the volume of the expected suspension of liposomes. Using a rotary evaporator, the solvent is removed at approximately 40° C. under negative pressure. The solvent normally is removed within about 5 min to 2 h, depending on the desired volume of the liposomes. The composition can be dried further in a desiccator under vacuum. The dried lipids generally are discarded after about 1 week because of a tendency to deteriorate with time.

Dried lipids can be hydrated at approximately 25-50 mM phospholipid in sterile, pyrogen-free water by shaking until all the lipid film is resuspended. The aqueous liposomes can be then separated into aliquots, each placed in a vial, lyophilized and sealed under vacuum.

The dried lipids or lyophilized liposomes prepared as described above may be dehydrated and reconstituted in a solution of a protein or peptide and diluted to an appropriate concentration with a suitable solvent, e.g., DPBS. The mixture is then vigorously shaken in a vortex mixer. Unencapsulated additional materials, such as agents including but not limited to hormones, drugs, nucleic acid constructs and the like, are removed by centrifugation at 29,000× g and the liposomal pellets washed. The washed liposomes are resuspended at an appropriate total phospholipid concentration, e.g., about 50-200 mM. The amount of additional material or active agent encapsulated can be determined in accordance with standard methods. After determination of the amount of additional material or active agent encapsulated in the liposome preparation, the liposomes may be diluted to appropriate concentrations and stored at 4° C. until use. A pharmaceutical composition comprising the liposomes will usually include a sterile, pharmaceutically acceptable carrier or diluent, such as water or saline solution.

Additional liposomes which may be useful with the present embodiments include cationic liposomes, for example, as described in WO02/100435A1, U.S. Pat. No. 5,962,016, U.S. Application 2004/0208921, WO03/015757A1, WO04029213A2, U.S. Pat. Nos. 5,030,453, and 6,680,068, all of which are hereby incorporated by reference in their entirety without disclaimer.

In preparing such liposomes, any protocol described herein, or as would be known to one of ordinary skill in the art may be used. Additional non-limiting examples of preparing liposomes are described in U.S. Pat. Nos. 4,728,578, 4,728,575, 4,737,323, 4,533,254, 4,162,282, 4,310,505, and 4,921,706; WO1986/000238 and WO1990/004943, each incorporated herein by reference.

In certain embodiments, the lipid based nanoparticle is a neutral liposome (e.g., a DOPC liposome). “Neutral liposomes” or “non-charged liposomes”, as used herein, are defined as liposomes having one or more lipid components that yield an essentially-neutral, net charge (substantially non-charged). By “essentially neutral” or “essentially non-charged”, it is meant that few, if any, lipid components within a given population (e.g., a population of liposomes) include a charge that is not canceled by an opposite charge of another component (i.e., fewer than 10% of components include a non-canceled charge, more preferably fewer than 5%, and most preferably fewer than 1%). In certain embodiments, neutral liposomes may include mostly lipids and/or phospholipids that are themselves neutral under physiological conditions (i.e., at about pH 7).

Liposomes and/or lipid-based nanoparticles of the present embodiments may comprise a phospholipid. In certain embodiments, a single kind of phospholipid may be used in the creation of liposomes (e.g., a neutral phospholipid, such as DOPC, may be used to generate neutral liposomes). In other embodiments, more than one kind of phospholipid may be used to create liposomes. Phospholipids may be from natural or synthetic sources. Phospholipids include, for example, phosphatidylcholines, phosphatidylglycerols, and phosphatidylethanolamines; because phosphatidylethanolamines and phosphatidyl cholines are non-charged under physiological conditions (i.e., at about pH 7), these compounds may be particularly useful for generating neutral liposomes. In certain embodiments, the phospholipid DOPC is used to produce non-charged liposomes. In certain embodiments, a lipid that is not a phospholipid (e.g., a cholesterol) may be used

Phospholipids include glycerophospholipids and certain sphingolipids. Phospholipids include, but are not limited to, dioleoylphosphatidylycholine (“DOPC”), egg phosphatidylcholine (“EPC”), dilauryloylphosphatidylcholine (“DLPC”), dimyristoylphosphatidylcholine (“DMPC”), dipalmitoylphosphatidylcholine (“DPPC”), distearoylphosphatidylcholine (“DSPC”), 1-myristoyl-2-palmitoyl phosphatidylcholine (“MPPC”), 1-palmitoyl-2-myristoyl phosphatidylcholine (“PMPC”), 1-palmitoyl-2-stearoyl phosphatidylcholine (“PSPC”), 1-stearoyl-2-palmitoyl phosphatidylcholine (“SPPC”), dilauryloylphosphatidylglycerol (“DLPG”), dimyristoylphosphatidylglycerol (“DMPG”), dipalmitoylphosphatidylglycerol (“DPPG”), distearoylphosphatidylglycerol (“DSPG”), distearoyl sphingomyelin (“DSSP”), distearoylphophatidylethanolamine (“DSPE”), dioleoylphosphatidylglycerol (“DOPG”), dimyristoyl phosphatidic acid (“DMPA”), dipalmitoyl phosphatidic acid (“DPPA”), dimyristoyl phosphatidylethanolamine (“DMPE”), dipalmitoyl phosphatidylethanolamine (“DPPE”), dimyristoyl phosphatidylserine (“DMPS”), dipalmitoyl phosphatidylserine (“DPPS”), brain phosphatidylserine (“BPS”), brain sphingomyelin (“BSP”), dipalmitoyl sphingomyelin (“DPSP”), dimyristyl phosphatidylcholine (“DMPC”), 1,2-distearoyl-sn-glycero-3-phosphocholine (“DAPC”), 1,2-diarachidoyl-sn-glycero-3-phosphocholine (“DBPC”), 1,2-dieicosenoyl-sn-glycero-3-phosphocholine (“DEPC”), dioleoylphosphatidylethanolamine (“DOPE”), palmitoyloeoyl phosphatidylcholine (“POPC”), palmitoyloeoyl phosphatidylethanolamine (“POPE”), lysophosphatidylcholine, lysophosphatidylethanolamine, and dilinoleoylphosphatidylcholine.

B. Exosomes

“Extracellular vesicles” and “EVs” are cell-derived and cell-secreted microvesicles which, as a class, include exosomes, exosome-like vesicles, ectosomes (which result from budding of vesicles directly from the plasma membrane), microparticles, microvesicles, shedding microvesicles (SMVs), nanoparticles and even (large) apoptotic blebs or bodies (resulting from cell death) or membrane particles.

The terms “microvesicle” and “exosomes,” as used herein, refer to a membranous particle having a diameter (or largest dimension where the particles is not spheroid) of between about 10 nm to about 5000 nm, more typically between 30 nm and 1000 nm, and most typically between about 50 nm and 750 nm, wherein at least part of the membrane of the exosomes is directly obtained from a cell. Most commonly, exosomes will have a size (average diameter) that is up to 5% of the size of the donor cell. Therefore, especially contemplated exosomes include those that are shed from a cell.

Exosomes may be detected in or isolated from any suitable sample type, such as, for example, body fluids. As used herein, the term “isolated” refers to separation out of its natural environment and is meant to include at least partial purification and may include substantial purification. As used herein, the term “sample” refers to any sample suitable for the methods provided by the present invention. The sample may be any sample that includes exosomes suitable for detection or isolation. Sources of samples include blood, bone marrow, pleural fluid, peritoneal fluid, cerebrospinal fluid, urine, saliva, amniotic fluid, malignant ascites, broncho-alveolar lavage fluid, synovial fluid, breast milk, sweat, tears, joint fluid, and bronchial washes. In one aspect, the sample is a blood sample, including, for example, whole blood or any fraction or component thereof. A blood sample suitable for use with the present invention may be extracted from any source known that includes blood cells or components thereof, such as venous, arterial, peripheral, tissue, cord, and the like. For example, a sample may be obtained and processed using well-known and routine clinical methods (e.g., procedures for drawing and processing whole blood). In one aspect, an exemplary sample may be peripheral blood drawn from a subject with cancer.

Exosomes may also be isolated from tissue samples, such as surgical samples, biopsy samples, tissues, feces, and cultured cells. When isolating exosomes from tissue sources it may be necessary to homogenize the tissue in order to obtain a single cell suspension followed by lysis of the cells to release the exosomes. When isolating exosomes from tissue samples it is important to select homogenization and lysis procedures that do not result in disruption of the exosomes. Exosomes contemplated herein are preferably isolated from body fluid in a physiologically acceptable solution, for example, buffered saline, growth medium, various aqueous medium, etc.

Exosomes may be isolated from freshly collected samples or from samples that have been stored frozen or refrigerated. In some embodiments, exosomes may be isolated from cell culture medium. Although not necessary, higher purity exosomes may be obtained if fluid samples are clarified before precipitation with a volume-excluding polymer, to remove any debris from the sample. Methods of clarification include centrifugation, ultracentrifugation, filtration, or ultrafiltration. Most typically, exosomes can be isolated by numerous methods well-known in the art. One preferred method is differential centrifugation from body fluids or cell culture supernatants. Exemplary methods for isolation of exosomes are described in (Losche et al., 2004; Mesri and Altieri, 1998; Morel et al., 2004). Alternatively, exosomes may also be isolated via flow cytometry as described in (Combes et al., 1997).

One accepted protocol for isolation of exosomes includes ultracentrifugation, often in combination with sucrose density gradients or sucrose cushions to float the relatively low-density exosomes. Isolation of exosomes by sequential differential centrifugations is complicated by the possibility of overlapping size distributions with other microvesicles or macromolecular complexes. Furthermore, centrifugation may provide insufficient means to separate vesicles based on their sizes. However, sequential centrifugations, when combined with sucrose gradient ultracentrifugation, can provide high enrichment of exosomes.

Isolation of exosomes based on size, using alternatives to the ultracentrifugation routes, is another option. Successful purification of exosomes using ultrafiltration procedures that are less time consuming than ultracentrifugation, and do not require use of special equipment have been reported. Similarly, a commercial kit is available (EXOMIR™, Bioo Scientific) which allows removal of cells, platelets, and cellular debris on one microfilter and capturing of vesicles bigger than 30 nm on a second microfilter using positive pressure to drive the fluid. However, for this process, the exosomes are not recovered, their RNA content is directly extracted from the material caught on the second microfilter, which can then be used for PCR analysis. HPLC-based protocols could potentially allow one to obtain highly pure exosomes, though these processes require dedicated equipment and are difficult to scale up. A significant problem is that both blood and cell culture media contain large numbers of nanoparticles (some non-vesicular) in the same size range as exosomes. For example, some miRNAs may be contained within extracellular protein complexes rather than exosomes; however, treatment with protease (e.g., proteinase K) can be performed to eliminate any possible contamination with “extraexosomal” protein.

In another embodiment, cancer cell-derived exosomes may be captured by techniques commonly used to enrich a sample for exosomes, such as those involving immunospecific interactions (e.g., immunomagnetic capture) Immunomagnetic capture, also known as immunomagnetic cell separation, typically involves attaching antibodies directed to proteins found on a particular cell type to small paramagnetic beads. When the antibody-coated beads are mixed with a sample, such as blood, they attach to and surround the particular cell. The sample is then placed in a strong magnetic field, causing the beads to pellet to one side. After removing the blood, captured cells are retained with the beads. Many variations of this general method are well-known in the art and suitable for use to isolate exosomes. In one example, the exosomes may be attached to magnetic beads (e.g., aldehyde/sulphate beads) and then an antibody is added to the mixture to recognize an epitope on the surface of the exosomes that are attached to the beads. Exemplary proteins that are known to be found on cancer cell-derived exosomes include ATP-binding cassette sub-family A member 6 (ABCA6), tetraspanin-4 (TSPAN4), SLIT and NTRK-like protein 4 (SLITRK4), putative protocadherin beta-18 (PCDHB18), myeloid cell surface antigen CD33 (CD33), and glypican-1 (GPC1). Cancer cell-derived exosomes may be isolated using, for example, antibodies or aptamers to one or more of these proteins.

As used herein, analysis includes any method that allows direct or indirect visualization of exosomes and may be in vivo or ex vivo. For example, analysis may include, but not limited to, ex vivo microscopic or cytometric detection and visualization of exosomes bound to a solid substrate, flow cytometry, fluorescent imaging, and the like. In an exemplary aspect, cancer cell-derived exosomes are detected using antibodies directed to one or more of ATP-binding cassette sub-family A member 6 (ABCA6), tetraspanin-4 (TSPAN4), SLIT and NTRK-like protein 4 (SLITRK4), putative protocadherin beta-18 (PCDHB18), myeloid cell surface antigen CD33 (CD33), glypican-1 (GPC1), Histone H2A type 2-A (HIST1H2AA), Histone H2A type 1-A (HIST1H1AA), Histone H3.3 (H3F3A), Histone H3.1 (HIST1H3A), Zinc finger protein 37 homolog (ZFP37), Laminin subunit beta-1 (LAMB1), Tubulointerstitial nephritis antigen-like (TINAGL1), Peroxiredeoxin-4 (PRDX4), Collagen alpha-2(IV) chain (COL4A2), Putative protein C3P1 (C3P1), Hemicentin-1 (HMCN1), Putative rhophilin-2-like protein (RHPN2P1), Ankyrin repeat domain-containing protein 62 (ANKRD62), Tripartite motif-containing protein 42 (TRIM42), Junction plakoglobin (JUP), Tubulin beta-2B chain (TUBB2B), Endoribonuclease Dicer (DICER1), E3 ubiquitin-protein ligase TRIM71 (TRIM71), Katanin p60 ATPase-containing subunit A-like 2 (KATNAL2), Protein S100-A6 (S100A6), 5′-nucleotidase domain-containing protein 3 (NT5DC3), Valine-tRNA ligase (VARS), Kazrin (KAZN), ELAV-like protein 4 (ELAVL4), RING finger protein 166 (RNF166), FERM and PDZ domain-containing protein 1 (FRMPD1), 78 kDa glucose-regulated protein (HSPA5), Trafficking protein particle complex subunit 6A (TRAPPC6A), Squalene monooxygenase (SQLE), Tumor susceptibility gene 101 protein (TSG101), Vacuolar protein sorting 28 homolog (VPS28), Prostaglandin F2 receptor negative regulator (PTGFRN), Isobutyryl-CoA dehydrogenase, mitochondrial (ACAD8), 26S protease regulatory subunit 6B (PSMC4), Elongation factor 1-gamma (EEF1G), Titin (TTN), Tyrosine-protein phosphatase type 13 (PTPN13), Triosephosphate isomerase (TPI1), or Carboxypeptidase E (CPE) and subsequently bound to a solid substrate and/or visualized using microscopic or cytometric detection.

It should be noted that not all proteins expressing in a cell are found in exosomes secreted by that cell. For example, calnexin, GM130, and LAMP-2 are all proteins expressed in MCF-7 cells but not found in exosomes secreted by MCF-7 cells (Baietti et al., 2012). As another example, one study found that 190/190 pancreatic ductal adenocarcinoma patients had higher levels of GPC1+ exosomes than healthy controls (Melo et al., 2015, which is incorporated herein by reference in its entirety). Notably, only 2.3% of healthy controls, on average, had GPC1+ exosomes.

1. Exemplary Protocol for Collecting Exosomes from Cell Culture

On Day 1, seed enough cells (e.g., about five million cells) in T225 flasks in media containing 10% FBS so that the next day the cells will be about 70% confluent. On Day 2, aspirate the media on the cells, wash the cells twice with PBS, and then add 25-30 mL base media (i.e., no PenStrep or FBS) to the cells. Incubate the cells for 24-48 hours. A 48 hour incubation is preferred, but some cells lines are more sensitive to serum-free media and so the incubation time should be reduced to 24 hours. Note that FBS contains exosomes that will heavily skew NanoSight results.

On Day 3/4, collect the media and centrifuge at room temperature for five minutes at 800× g to pellet dead cells and large debris. Transfer the supernatant to new conical tubes and centrifuge the media again for 10 minutes at 2000× g to remove other large debris and large vesicles. Pass the media through a 0.2 μm filter and then aliquot into ultracentrifuge tubes (e.g., 25×89 mm Beckman Ultra-Clear) using 35 mL per tube. If the volume of media per tube is less than 35 mL, fill the remainder of the tube with PBS to reach 35 mL. Ultracentrifuge the media for 2-4 hours at 28,000 rpm at 4° C. using a SW 32 Ti rotor (k-factor 266.7, RCF max 133,907). Carefully aspirate the supernatant until there is roughly 1-inch of liquid remaining. Tilt the tube and allow remaining media to slowly enter aspirator pipette. If desired, the exosomes pellet can be resuspended in PBS and the ultracentrifugation at 28,000 rpm repeated for 1-2 hours to further purify the population of exosomes.

Finally, resuspend the exosomes pellet in 210 μL PBS. If there are multiple ultracentrifuge tubes for each sample, use the same 210 μL PBS to serially resuspend each exosomes pellet. For each sample, take 10 μL and add to 990 μL H₂O to use for nanoparticle tracking analysis. Use the remaining 200 μL exosomes-containing suspension for downstream processes or immediately store at −80° C.

2. Exemplary Protocol for Extracting Exosomes from Serum Samples

First, allow serum samples to thaw on ice. Then, dilute 250 μL of cell-free serum samples in 11 mL PBS; filter through a 0.2 μm pore filter. Ultracentrifuge the diluted sample at 150,000×g overnight at 4° C. The following day, carefully discard the supernatant and wash the exosomes pellet in 11 mL PBS. Perform a second round of ultracentrifugation at 150,000×g at 4° C. for 2 hours. Finally, carefully discard the supernatant and resuspend the exosomes pellet in 100 μL PBS for analysis.

C. Exemplary Protocol for Electroporation of Exosomes and Liposomes

Mix 1×10⁸ exosomes (measured by NanoSight analysis) or 100 nm liposomes (e.g., purchased from Encapsula Nano Sciences) and 1 μg of siRNA (Qiagen) or shRNA in 400 μL of electroporation buffer (1.15 mM potassium phosphate, pH 7.2, 25 mM potassium chloride, 21% Optiprep). Electroporate the exosomes or liposomes using a 4 mm cuvette (see, e.g., Alvarez-Erviti et al., 2011; El-Andaloussi et al., 2012). After electroporation, treat the exosomes or liposomes with protease-free RNAse followed by addition of 10× concentrated RNase inhibitor. Finally, wash the exosomes or liposomes with PBS under ultracentrifugation methods, as described above.

### II. CRISPR/CAS SYSTEMS

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). One or more elements of a CRISPR system can derive from a type I, type II, or type III CRISPR system, e.g., derived from a particular organism comprising an endogenous CRISPR system, such as Streptococcus pyogenes.

In some aspects, a Cas nuclease and gRNA (including a fusion of crRNA specific for the target sequence and fixed tracrRNA) are introduced into the cell. In general, target sites at the 5′ end of the gRNA target the Cas nuclease to the target site, e.g., the gene, using complementary base pairing. The target site may be selected based on its location immediately 5′ of a protospacer adjacent motif (PAM) sequence, such as typically NGG, or NAG. In this respect, the gRNA is targeted to the desired sequence by modifying the first 20, 19, 18, 17, 16, 15, 14, 14, 12, 11, or 10 nucleotides of the guide RNA to correspond to the target DNA sequence. In general, a CRISPR system is characterized by elements that promote the formation of a CRISPR complex at the site of a target sequence. Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex.

The CRISPR system can induce double stranded breaks (DSBs) at the target site, followed by disruptions as discussed herein. In other embodiments, Cas9 variants, deemed “nickases,” are used to nick a single strand at the target site. Paired nickases can be used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a transcriptional repressor or activator, to affect gene expression.

The target sequence may comprise any polynucleotide, such as DNA or RNA polynucleotides. The target sequence may be located in the nucleus or cytoplasm of the cell, such as within an organelle of the cell. Generally, a sequence or template that may be used for recombination into the targeted locus comprising the target sequences is referred to as an “editing template” or “editing polynucleotide” or “editing sequence”. In some aspects, an exogenous template polynucleotide may be referred to as an editing template. In some aspects, the recombination is homologous recombination.

Typically, in the context of an endogenous CRISPR system, formation of the CRISPR complex (comprising the guide sequence hybridized to the target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. The tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of the CRISPR complex, such as by hybridization along at least a portion of the tracr sequence to all or a portion of a tracr mate sequence that is operably linked to the guide sequence. The tracr sequence has sufficient complementarity to a tracr mate sequence to hybridize and participate in formation of the CRISPR complex, such as at least 50%, 60%, 70%, 80%, 90%, 95% or 99% of sequence complementarity along the length of the tracr mate sequence when optimally aligned.

One or more vectors driving expression of one or more elements of the CRISPR system can be introduced into the cell such that expression of the elements of the CRISPR system direct formation of the CRISPR complex at one or more target sites. Components can also be delivered to cells as proteins and/or RNA. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. The vector may comprise one or more insertion sites, such as a restriction endonuclease recognition sequence (also referred to as a “cloning site”). In some embodiments, one or more insertion sites are located upstream and/or downstream of one or more sequence elements of one or more vectors. When multiple different guide sequences are used, a single expression construct may be used to target CRISPR activity to multiple different, corresponding target sequences within a cell.

A vector may comprise a regulatory element operably linked to an enzyme-coding sequence encoding the CRISPR enzyme, such as a Cas protein. Non-limiting examples of Cas proteins include Cast, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2.

The CRISPR enzyme can be Cas9 (e.g., from S. pyogenes or S. pneumonia). The CRISPR enzyme can direct cleavage of one or both strands at the location of a target sequence, such as within the target sequence and/or within the complement of the target sequence. The vector can encode a CRISPR enzyme that is mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce NHEJ or HDR.

In some embodiments, an enzyme coding sequence encoding the CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

In general, a guide sequence is any polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more.

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), Clustal W, Clustal X, BLAT, Novoalign (Novocraft Technologies, ELAND (Illumina, San Diego, Calif.), SOAP (available at soap.genomics.org.cn), and Maq (available at maq.sourceforge.net).

The CRISPR enzyme may be part of a fusion protein comprising one or more heterologous protein domains. A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions. Additional domains that may form part of a fusion protein comprising a CRISPR enzyme are described in US 20110059502, incorporated herein by reference.

### III. DELIVERY OF THE CRISPR SYSTEM

In some aspects, a nucleic acid encoding the CRISPR-Cas9 targeting molecule, complex, or combination, is administered or introduced to the cell. In some aspects, the system may already be present in the cell, or within exosomes in cell. The nucleic acid typically is administered in the form of an expression vector, such as a viral expression vector. In some aspects, the expression vector is a retroviral expression vector, an adenoviral expression vector, a DNA plasmid expression vector, or an AAV expression vector. In some aspects, one or more polynucleotides encoding the disruption molecule or complex, such as the DNA-targeting molecule, is delivered to the cell. In some aspects, the delivery is by delivery of one or more vectors, one or more transcripts thereof, and/or one or more proteins transcribed therefrom, is delivered to the cell.

In some embodiments, the polypeptides are synthesized in situ in the cell as a result of the introduction of polynucleotides encoding the polypeptides into the cell. In some aspects, the polypeptides could be produced outside the cell and then introduced thereto. Methods for introducing a polynucleotide construct into animal cells are known and include, as non-limiting examples stable transformation methods wherein the polynucleotide construct is integrated into the genome of the cell, transient transformation methods wherein the polynucleotide construct is not integrated into the genome of the cell, and virus mediated methods. In some embodiments, the polynucleotides may be introduced into the cell by for example, recombinant viral vectors (e.g. retroviruses, adenoviruses), liposome and the like. For example, in some aspects, transient transformation methods include microinjection, electroporation, or particle bombardment. In some embodiments, the polynucleotides may be included in vectors, more particularly plasmids or virus, in view of being expressed in the cells.

In some embodiments, viral and non-viral based gene transfer methods can be used to introduce nucleic acids in mammalian cells or target tissues. Such methods can be used to administer nucleic acids encoding components of a CRISPR system to cells in culture, or in a host organism. Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. For a review of gene therapy procedures, see Anderson, 1992; Nabel & Feigner, 1993; Mitani & Caskey, 1993; Dillon, 1993; Miller, 1992; Van Brunt, 1988; Vigne, 1995; Kremer & Perricaudet, 1995; Haddada et al., 1995; and Yu et al., 1994.

Methods of non-viral delivery of nucleic acids include exosomes, lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in (e.g., U.S. Pat. Nos. 5,049,386, 4,946,787; and 4,897,355) and lipofection reagents are sold commercially (e.g., Transfectam™ and Lipofectin™) Cationic and neutral lipids that are suitable for efficient receptor-recognition lipofection of polynucleotides include those of Feigner, WO 91117424; WO 91116024. Delivery can be to cells (e.g. in vitro or ex vivo administration) or target tissues (e.g. in vivo administration).

In some embodiments, delivery is via the use of RNA or DNA viral based systems for the delivery of nucleic acids. Viral vectors in some aspects may be administered directly to patients (in vivo) or they can be used to treat cells in vitro or ex vivo, and then administered to patients. Viral-based systems in some embodiments include retroviral, lentivirus, adenoviral, adeno-associated and herpes simplex virus vectors for gene transfer.

In some aspects, a reporter gene which includes but is not limited to glutathione-5-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP), may be introduced into the cell to encode a gene product which serves as a marker by which to measure the alteration or modification of expression of the gene product. In a further embodiment, the DNA molecule encoding the gene product may be introduced into the cell via a vector. In some embodiments, the gene product is luciferase.

As will be appreciated by one of skill in the art, prior or subsequent to loading with cargo, the present exosomes may be further altered by inclusion of a targeting moiety to enhance the utility thereof as a vehicle for delivery of cargo. In this regard, exosomes may be engineered to incorporate an entity that specifically targets a particular cell to tissue type. This target-specific entity, e.g. peptide having affinity for a receptor or ligand on the target cell or tissue, may be integrated within the exosomal membrane, for example, by fusion to an exosomal membrane marker using methods well-established in the art.

### IV. TREATMENT OF DISEASES

Certain aspects of the present invention provide for treating a patient with exosomes that express or comprise a gene editing system, such as a CRISPR system. The CRISPR system may induce gene editing within cancer cells in the patient. As exosomes are known to comprise the machinery necessary to complete mRNA transcription and protein translation (see WO2015/085096, which is incorporated herein by reference in its entirety), mRNA or DNA nucleic acids encoding a therapeutic protein may be transfected into exosomes. Alternatively, the therapeutic protein itself may be electroporated into the exosomes or incorporated directly into a liposome.

The term “subject” as used herein refers to any individual or patient to which the subject methods are performed. Generally the subject is human, although as will be appreciated by those in the art, the subject may be an animal. Thus other animals, including mammals, such as rodents (including mice, rats, hamsters, and guinea pigs), cats, dogs, rabbits, farm animals (including cows, horses, goats, sheep, pigs, etc.), and primates (including monkeys, chimpanzees, orangutans, and gorillas) are included within the definition of subject.

“Treatment” and “treating” refer to administration or application of a therapeutic agent to a subject or performance of a procedure or modality on a subject for the purpose of obtaining a therapeutic benefit of a disease or health-related condition. For example, a treatment may include administration of exosomes comprising a CRISPR system, chemotherapy, immunotherapy, or radiotherapy, performance of surgery, or any combination thereof.

The term “therapeutic benefit” or “therapeutically effective” as used herein refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the invasiveness of a tumor, reduction in the growth rate of the cancer, or prevention of metastasis. Treatment of cancer may also refer to prolonging survival of a subject with cancer.

The term “cancer,” as used herein, may be used to describe a solid tumor, metastatic cancer, or non-metastatic cancer. In certain embodiments, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. Nonetheless, it is also recognized that the present invention may also be used to treat a non-cancerous disease (e.g., a fungal infection, a bacterial infection, a viral infection, a neurodegenerative disease, and/or a genetic disorder).

The terms “contacted” and “exposed,” when applied to a cell, are used herein to describe the process by which a therapeutic agent is delivered to a target cell or are placed in direct juxtaposition with the target cell. To achieve cell killing, for example, one or more agents are delivered to a cell in an amount effective to kill the cell or prevent it from dividing.

An effective response of a patient or a patient's “responsiveness” to treatment refers to the clinical or therapeutic benefit imparted to a patient at risk for, or suffering from, a disease or disorder. Such benefit may include cellular or biological responses, a complete response, a partial response, a stable disease (without progression or relapse), or a response with a later relapse. For example, an effective response can be reduced tumor size or progression-free survival in a patient diagnosed with cancer.

Treatment outcomes can be predicted and monitored and/or patients benefiting from such treatments can be identified or selected via the methods described herein.

Regarding neoplastic condition treatment, depending on the stage of the neoplastic condition, neoplastic condition treatment involves one or a combination of the following therapies: surgery to remove the neoplastic tissue, radiation therapy, and chemotherapy. Other therapeutic regimens may be combined with the administration of the anticancer agents, e.g., therapeutic compositions and chemotherapeutic agents. For example, the patient to be treated with such anti-cancer agents may also receive radiation therapy and/or may undergo surgery.

For the treatment of disease, the appropriate dosage of a therapeutic composition will depend on the type of disease to be treated, as defined above, the severity and course of the disease, the patient's clinical history and response to the agent, and the discretion of the attending physician. The agent is suitably administered to the patient at one time or over a series of treatments.

Therapeutic and prophylactic methods and compositions can be provided in a combined amount effective to achieve the desired effect. A tissue, tumor, or cell can be contacted with one or more compositions or pharmacological formulation(s) comprising one or more of the agents, or by contacting the tissue, tumor, and/or cell with two or more distinct compositions or formulations. Also, it is contemplated that such a combination therapy can be used in conjunction with chemotherapy, radiotherapy, surgical therapy, or immunotherapy.

Administration in combination can include simultaneous administration of two or more agents in the same dosage form, simultaneous administration in separate dosage forms, and separate administration. That is, the subject therapeutic composition and another therapeutic agent can be formulated together in the same dosage form and administered simultaneously. Alternatively, subject therapeutic composition and another therapeutic agent can be simultaneously administered, wherein both the agents are present in separate formulations. In another alternative, the therapeutic agent can be administered just followed by the other therapeutic agent or vice versa. In the separate administration protocol, the subject therapeutic composition and another therapeutic agent may be administered a few minutes apart, or a few hours apart, or a few days apart.

A first anti-cancer treatment (e.g., exosomes that express a recombinant protein or with a recombinant protein isolated from exosomes) may be administered before, during, after, or in various combinations relative to a second anti-cancer treatment. The administrations may be in intervals ranging from concurrently to minutes to days to weeks. In embodiments where the first treatment is provided to a patient separately from the second treatment, one would generally ensure that a significant period of time did not expire between the time of each delivery, such that the two compounds would still be able to exert an advantageously combined effect on the patient. In such instances, it is contemplated that one may provide a patient with the first therapy and the second therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

In certain embodiments, a course of treatment will last 1-90 days or more (this such range includes intervening days). It is contemplated that one agent may be given on any day of day 1 to day 90 (this such range includes intervening days) or any combination thereof, and another agent is given on any day of day 1 to day 90 (this such range includes intervening days) or any combination thereof. Within a single day (24-hour period), the patient may be given one or multiple administrations of the agent(s). Moreover, after a course of treatment, it is contemplated that there is a period of time at which no anti-cancer treatment is administered. This time period may last 1-7 days, and/or 1-5 weeks, and/or 1-12 months or more (this such range includes intervening days), depending on the condition of the patient, such as their prognosis, strength, health, etc. It is expected that the treatment cycles would be repeated as necessary.

Various combinations may be employed. For the example below a first anti-cancer therapy is “A” and a second anti-cancer therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of the present invention to a patient will follow general protocols for the administration of such compounds, taking into account the toxicity, if any, of the agents. Therefore, in some embodiments there is a step of monitoring toxicity that is attributable to combination therapy.

1. Chemotherapy

A wide variety of chemotherapeutic agents may be used in accordance with the present invention. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammall and calicheamicin omegaI1); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PS Kpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine, plicomycin, gemcitabien, navelbine, farnesyl-protein tansferase inhibitors, transplatinum, and pharmaceutically acceptable salts, acids, or derivatives of any of the above.

2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

3. Immunotherapy

The skilled artisan will understand that additional immunotherapies may be used in combination or in conjunction with methods of the invention. In the context of cancer treatment, immunotherapeutics, generally, rely on the use of immune effector cells and molecules to target and destroy cancer cells. Rituximab (Rituxan®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

In one aspect of immunotherapy, the tumor cell must bear some marker that is amenable to targeting, i.e., is not present on the majority of other cells. Many tumor markers exist and any of these may be suitable for targeting in the context of the present invention. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons α, β, and γ, IL-1, GM-CSF, and TNF (Bukowski et al., 1998; Davidson et al., 1998; Hellstrand et al., 1998); gene therapy, e.g., TNF, IL-1, IL-2, and p53 (Qin et al., 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2013; Hanibuchi et al., 1998; U.S. Pat. No. 5,824,311). It is contemplated that one or more anti-cancer therapies may be employed with the antibody therapies described herein.

In some embodiments, the immunotherapy may be an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAGS), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies (e.g., International Patent Publication WO2015016718; Pardoll, Nat Rev Cancer, 12(4): 252-64, 2012; both incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present disclosure. For example, it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 axis antagonists for use in the methods provided herein are known in the art such as described in U.S. Patent Publication Nos. 20140294898, 2014022021, and 20110008369, all incorporated herein by reference.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PDL2-Fc fusion soluble receptor described in WO2010/027827 and WO2011/066342.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al. (1998) Proc Natl Acad Sci USA 95(17): 10067-10071; Camacho et al. (2004) J Clin Oncology 22(145): Abstract No. 2505 (antibody CP-675206); and Mokyr et al. (1998) Cancer Res 58:5301-5304 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001014424, WO2000037504, and U.S. Pat. No. 8,017,114; all incorporated herein by reference.

An exemplary anti-CTLA-4 antibody is ipilimumab (also known as 10D1, MDX-010, MDX-101, and Yervoy®) or antigen binding fragments and variants thereof (see, e.g., WO 01/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

Other molecules for modulating CTLA-4 include CTLA-4 ligands and receptors such as described in U.S. Pat. Nos. 5,844,905, 5,885,796 and International Patent Application Nos. WO1995001994 and WO1998042752; all incorporated herein by reference, and immunoadhesins such as described in U.S. Pat. No. 8,329,867, incorporated herein by reference.

In some embodiment, the immune therapy could be adoptive immunotherapy, which involves the transfer of autologous antigen-specific T cells generated ex vivo. The T cells used for adoptive immunotherapy can be generated either by expansion of antigen-specific T cells or redirection of T cells through genetic engineering (Park, Rosenberg et al. 2011). Isolation and transfer of tumor specific T cells has been shown to be successful in treating melanoma. Novel specificities in T cells have been successfully generated through the genetic transfer of transgenic T cell receptors or chimeric antigen receptors (CARs) (Jena, Dotti et al. 2010). CARs are synthetic receptors consisting of a targeting moiety that is associated with one or more signaling domains in a single fusion molecule. In general, the binding moiety of a CAR consists of an antigen-binding domain of a single-chain antibody (scFv), comprising the light and variable fragments of a monoclonal antibody joined by a flexible linker. Binding moieties based on receptor or ligand domains have also been used successfully. The signaling domains for first generation CARs are derived from the cytoplasmic region of the CD3zeta or the Fc receptor gamma chains. CARs have successfully allowed T cells to be redirected against antigens expressed at the surface of tumor cells from various malignancies including lymphomas and solid tumors (Jena, Dotti et al. 2010).

In one embodiment, the present application provides for a combination therapy for the treatment of cancer wherein the combination therapy comprises adoptive T cell therapy and a checkpoint inhibitor. In one aspect, the adoptive T cell therapy comprises autologous and/or allogenic T-cells. In another aspect, the autologous and/or allogenic T-cells are targeted against tumor antigens.

4. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present invention, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

5. Other Agents

It is contemplated that other agents may be used in combination with certain aspects of the present invention to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present invention to improve the anti-hyperproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present invention to improve the treatment efficacy.

### V. PHARMACEUTICAL COMPOSITIONS

It is contemplated that exosomes that express or comprise a CRISPR system can be administered systemically or locally to inhibit tumor cell growth and, most preferably, to kill cancer cells in cancer patients with locally advanced or metastatic cancers. They can be administered intravenously, intrathecally, and/or intraperitoneally. They can be administered alone or in combination with anti-proliferative drugs. In one embodiment, they are administered to reduce the cancer load in the patient prior to surgery or other procedures. Alternatively, they can be administered after surgery to ensure that any remaining cancer (e.g., cancer that the surgery failed to eliminate) does not survive.

It is not intended that the present invention be limited by the particular nature of the therapeutic preparation. For example, such compositions can be provided in formulations together with physiologically tolerable liquid, gel, solid carriers, diluents, or excipients. These therapeutic preparations can be administered to mammals for veterinary use, such as with domestic animals, and clinical use in humans in a manner similar to other therapeutic agents. In general, the dosage required for therapeutic efficacy will vary according to the type of use and mode of administration, as well as the particular requirements of individual subjects.

Where clinical applications are contemplated, it may be necessary to prepare pharmaceutical compositions comprising recombinant proteins and/or exosomes in a form appropriate for the intended application. Generally, pharmaceutical compositions, which can be parenteral formulations, can comprise an effective amount of one or more recombinant proteins and/or exosomes and/or additional agents dissolved or dispersed in a pharmaceutically acceptable carrier. The phrases “pharmaceutical or pharmacologically acceptable” refers to molecular entities and compositions that do not produce an adverse, allergic, or other untoward reaction when administered to an animal, such as, for example, a human, as appropriate. The preparation of a pharmaceutical composition comprising a recombinant protein and/or exosomes as disclosed herein, or additional active ingredients is as exemplified by Remington's Pharmaceutical Sciences, 18th Ed., 1990, which is incorporated herein by reference in its entirety for all purposes. Moreover, for animal (e.g., human) administration, it will be understood that preparations should meet sterility, pyrogenicity, general safety, and purity standards as required by the FDA Office of Biological Standards.

Further in accordance with certain aspects of the present invention, the composition suitable for administration may be provided in a pharmaceutically acceptable carrier with or without an inert diluent. As used herein, “pharmaceutically acceptable carrier” includes any and all aqueous solvents (e.g., water, alcoholic/aqueous solutions, ethanol, saline solutions, parenteral vehicles, such as sodium chloride, Ringer's dextrose, etc.), non-aqueous solvents (e.g., fats, oils, polyol (for example, glycerol, propylene glycol, and liquid polyethylene glycol, and the like), vegetable oil, and injectable organic esters, such as ethyloleate), lipids, liposomes, dispersion media, coatings (e.g., lecithin), surfactants, antioxidants, preservatives (e.g., antibacterial or antifungal agents, anti-oxidants, chelating agents, inert gases, parabens (e.g., methylparabens, propylparabens), chlorobutanol, phenol, sorbic acid, thimerosal or combinations thereof), isotonic agents (e.g., sugars and sodium chloride), absorption delaying agents (e.g., aluminum monostearate and gelatin), salts, drugs, drug stabilizers, gels, resins, fillers, binders, excipients, disintegration agents, lubricants, sweetening agents, flavoring agents, dyes, fluid and nutrient replenishers, such like materials and combinations thereof, as would be known to one of ordinary skill in the art. The carrier should be assimilable and includes liquid, semi-solid, i.e., pastes, or solid carriers. In addition, if desired, the compositions may contain minor amounts of auxiliary substances, such as wetting or emulsifying agents, stabilizing agents, or pH buffering agents. The pH and exact concentration of the various components in a pharmaceutical composition are adjusted according to well-known parameters. The proper fluidity can be maintained, for example, by the use of a coating, such as lecithin, by the maintenance of the required particle size in the case of dispersion, and by the use of surfactants.

A pharmaceutically acceptable carrier is particularly formulated for administration to a human, although in certain embodiments it may be desirable to use a pharmaceutically acceptable carrier that is formulated for administration to a non-human animal but that would not be acceptable (e.g., due to governmental regulations) for administration to a human Except insofar as any conventional carrier is incompatible with the active ingredient (e.g., detrimental to the recipient or to the therapeutic effectiveness of a composition contained therein), its use in the therapeutic or pharmaceutical compositions is contemplated. In accordance with certain aspects of the present invention, the composition is combined with the carrier in any convenient and practical manner, i.e., by solution, suspension, emulsification, admixture, encapsulation, absorption, and the like. Such procedures are routine for those skilled in the art.

Certain embodiments of the present invention may comprise different types of carriers depending on whether it is to be administered in solid, liquid, or aerosol form, and whether it needs to be sterile for the route of administration, such as injection. The compositions can be administered intravenously, intradermally, transdermally, intrathecally, intraarterially, intraperitoneally, intranasally, intravaginally, intrarectally, intramuscularly, subcutaneously, mucosally, orally, topically, locally, by inhalation (e.g., aerosol inhalation), by injection, by infusion, by continuous infusion, by localized perfusion bathing target cells directly, via a catheter, via a lavage, in lipid compositions (e.g., liposomes), or by other methods or any combination of the forgoing, which are described, for example, in Remington's Pharmaceutical Sciences, 18th Ed., 1990, incorporated herein by reference.

The active compounds can be formulated for parenteral administration, e.g., formulated for injection via the intravenous, intramuscular, sub-cutaneous, or even intraperitoneal routes. As such, the embodiments include parenteral formulations. Typically, such compositions can be prepared as either liquid solutions or suspensions; solid forms suitable for use to prepare solutions or suspensions upon the addition of a liquid prior to injection can also be prepared; and the preparations can also be emulsified.

According to the subject embodiments, the parenteral formulations can include exosomes as disclosed herein along with one or more solute and/or solvent, one or more buffering agent and/or one or more antimicrobial agents, or any combination thereof. In some aspects, the solvent can include water, water-miscible solvents, e.g., ethyl alcohol, liquid polyethylene glycol, and/or propylene glycol, and/or water-immiscible solvents, such as fixed oils including, for example, corn oil, cottonseed oil, peanut oil, and/or sesame oil. In certain versions, the solutes can include one or more antimicrobial agents, buffers, antioxidants, tonicity agents, cryoprotectants and/or lyoprotectants.

Antimicrobial agents according to the subject disclosure can include those provided elsewhere in the subject disclosure as well as benzyl alcohol, phenol, mercurials and/or parabens. Antimicrobial agents can include benzalkonium chloride, benzethonium chloride, benzyl alcohol, bronopol, centrimide, cetylpyridinium chloride, chlorhexidine, chlorobutanol, chlorocresol, chloroxylenol, cresol, ethyl alcohol, glycerin, exetidine, imidurea, phenol, phenoxyethanol, phenylethl alcohol, phenlymercuric nitrate, propylene glycol, and/or thimerosal, or any combination thereof. The antimicrobial agents can, in various aspects, be present in a concentration necessary to ensure sterility as is required for pharmaceutical agents. For example, the agents can be present in bacteriostatic or fungistatic concentrations in preparations, e.g., preparations contained in multiple-dose containers. The agents can, in various embodiments, be preservatives and/or can be present in adequate concentration at the time of use to prevent the multiplication of microorganisms, such as microorganisms inadvertently introduced into the preparation while, for example, withdrawing a portion of the contents with a hypodermic needle and syringe. In various aspects, the agents have maximum volume and/or concentration limits (e.g., phenylmercuric nitrate and thimerosal 0.01%, benzethonium chloride and benzalkonium chloride 0.01%, phenol or cresol 0.5%, and chlorobutanol 0.5%). In various instances, agents such as phenylmercuric nitrate, are employed in a concentration of 0.002%. Methyl p-hydroxybenzoate 0.18% and propyl p-hydroxybenzoate 0.02% in combination, and benzyl alcohol 2% also can be applied according to the embodiments. The antimicrobial agents can also include hexylresorcinol 0.5%, phenylmercuric benzoate 0.1%, and/or therapeutic compounds.

Antioxidants according to the subject disclosure can include ascorbic acid and/or its salts, and/or the sodium salt of ethylenediaminetetraacetic acid (EDTA). Tonicity agents as described herein can include electrolytes and/or mono- or disaccharides. Cryoprotectants and/or lyoprotectants are additives that protect biopharmaceuticals from detrimental effects due to freezing and/or drying of the product during freezedry processing. Cryoprotectants and/or lyoprotectants can include sugars (non-reducing) such as sucrose or trehalose, amino acids such as glycine or lysine, polymers such as liquid polyethylene glycol or dextran, and polyols such as mannitol or sorbitol all are possible cryo- or lyoprotectants. The subject embodiments can also include antifungal agents such as butyl paraben, methyl paraben, ethyl paraben, propyl paraben, benzoic acid, potassium sorbate, sodium benzoate, sodium propionate, and/or sorbic acid, or any combination thereof. Additional solutes and antimicrobial agents, buffers, antioxidants, tonicity agents, cryoprotectants and/or lyprotectants and characteristics thereof which may be employed according to the subject disclosure, as well as aspects of methods of making the subject parenteral formulations are described, for example, in Remington's Pharmaceutical Sciences, 21st Ed., 2005, e.g., Chapter 41, which is incorporated herein by reference in its entirety for all purposes.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The therapeutics may be formulated into a composition in a free base, neutral, or salt form. Pharmaceutically acceptable salts include the acid addition salts, e.g., those formed with the free amino groups of a proteinaceous composition, or which are formed with inorganic acids, such as, for example, hydrochloric or phosphoric acids, or such organic acids as acetic, oxalic, tartaric, or mandelic acid and the like. Salts formed with the free carboxyl groups can also be derived from inorganic bases, such as, for example, sodium, potassium, ammonium, calcium, or ferric hydroxides; or such organic bases as isopropylamine, trimethylamine, histidine, or procaine and the like. Upon formulation, solutions will be administered in a manner compatible with the dosage formulation and in such amount as is therapeutically effective. The formulations are easily administered in a variety of dosage forms, such as formulated for parenteral administrations, such as injectable solutions, or aerosols for delivery to the lungs, or formulated for alimentary administrations, such as drug release capsules and the like.

In a specific embodiment of the present invention, the composition is combined or mixed thoroughly with a semi-solid or solid carrier. The mixing can be carried out in any convenient manner, such as grinding. Stabilizing agents can be also added in the mixing process in order to protect the composition from loss of therapeutic activity, i.e., denaturation in the stomach. Examples of stabilizers for use in a composition include buffers, amino acids, such as glycine and lysine, carbohydrates, such as dextrose, mannose, galactose, fructose, lactose, sucrose, maltose, sorbitol, mannitol, etc.

In further embodiments, the present invention may concern the use of a pharmaceutical lipid vehicle composition comprising one or more lipids and an aqueous solvent. As used herein, the term “lipid” will be defined to include any of a broad range of substances that is characteristically insoluble in water and extractable with an organic solvent. This broad class of compounds is well known to those of skill in the art, and as the term “lipid” is used herein, it is not limited to any particular structure. Examples include compounds that contain long-chain aliphatic hydrocarbons and their derivatives. A lipid may be naturally occurring or synthetic (i.e., designed or produced by man) However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether- and ester-linked fatty acids, polymerizable lipids, and combinations thereof. Of course, compounds other than those specifically described herein that are understood by one of skill in the art as lipids are also encompassed by the compositions and methods.

One of ordinary skill in the art would be familiar with the range of techniques that can be employed for dispersing a composition in a lipid vehicle. For example, the therapeutic agent may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The term “unit dose” or “dosage” refers to physically discrete units suitable for use in a subject, each unit containing a predetermined quantity of the therapeutic composition calculated to produce the desired responses discussed above in association with its administration, i.e., the appropriate route and treatment regimen. The quantity to be administered, both according to number of treatments and unit dose, depends on the effect desired. The actual dosage amount of a composition of the present invention administered to a patient or subject can be determined by physical and physiological factors, such as body weight, the age, health, and sex of the subject, the type of disease being treated, the extent of disease penetration, previous or concurrent therapeutic interventions, idiopathy of the patient, the route of administration, and the potency, stability, and toxicity of the particular therapeutic substance. For example, a dose may also comprise from about 1 μg/kg/body weight to about 1000 mg/kg/body weight (this such range includes intervening doses) or more per administration, and any range derivable therein. In non-limiting examples of a derivable range from the numbers listed herein, a range of about 5 μg/kg/body weight to about 100 mg/kg/body weight, about 5 μg/kg/body weight to about 500 mg/kg/body weight, etc., can be administered. The practitioner responsible for administration will, in any event, determine the concentration of active ingredient(s) in a composition and appropriate dose(s) for the individual subject.

The actual dosage amount of a composition administered to an animal patient can be determined by physical and physiological factors, such as body weight, severity of condition, the type of disease being treated, previous or concurrent therapeutic interventions, idiopathy of the patient, and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, an active compound may comprise between about 2% to about 75% of the weight of the unit, or between about 25% to about 60%, for example, and any range derivable therein. Naturally, the amount of active compound(s) in each therapeutically useful composition may be prepared in such a way that a suitable dosage will be obtained in any given unit dose of the compound. Factors, such as solubility, bioavailability, biological half-life, route of administration, product shelf life, as well as other pharmacological considerations, will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 milligram/kg/body weight or more per administration, and any range derivable therein. In non-limiting examples of a derivable range from the numbers listed herein, a range of about 5 milligram/kg/body weight to about 100 milligram/kg/body weight, about 5 microgram/kg/body weight to about 500 milligram/kg/body weight, etc., can be administered, based on the numbers described above.

### VI. NUCLEIC ACIDS AND VECTORS

In certain aspects of the invention, nucleic acid sequences encoding a therapeutic protein or a fusion protein containing a therapeutic protein may be disclosed. Depending on which expression system is used, nucleic acid sequences can be selected based on conventional methods. For example, the respective genes or variants thereof may be codon optimized for expression in a certain system. Various vectors may be also used to express the protein of interest. Exemplary vectors include, but are not limited, plasmid vectors, viral vectors, transposon, or liposome-based vectors.

### VII. RECOMBINANT PROTEINS AND INHIBITORY RNAS

Some embodiments concern recombinant proteins and polypeptides. Particular embodiments concern a recombinant protein or polypeptide that had RNA-guided endonuclease activity. In further aspects, the protein or polypeptide may be modified to increase serum stability. Thus, when the present application refers to the function or activity of “modified protein” or a “modified polypeptide,” one of ordinary skill in the art would understand that this includes, for example, a protein or polypeptide that possesses an additional advantage over the unmodified protein or polypeptide. It is specifically contemplated that embodiments concerning a “modified protein” may be implemented with respect to a “modified polypeptide,” and vice versa.

Recombinant proteins may possess deletions and/or substitutions of amino acids; thus, a protein with a deletion, a protein with a substitution, and a protein with a deletion and a substitution are modified proteins. In some embodiments, these proteins may further include insertions or added amino acids, such as with fusion proteins or proteins with linkers, for example. A “modified deleted protein” lacks one or more residues of the native protein, but may possess the specificity and/or activity of the native protein. A “modified deleted protein” may also have reduced immunogenicity or antigenicity. An example of a modified deleted protein is one that has an amino acid residue deleted from at least one antigenic region that is, a region of the protein determined to be antigenic in a particular organism, such as the type of organism that may be administered the modified protein.

Substitution or replacement variants typically contain the exchange of one amino acid for another at one or more sites within the protein and may be designed to modulate one or more properties of the polypeptide, particularly its effector functions and/or bioavailability. Substitutions may or may not be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine, or methionine; serine to threonine; threonine to serine; tryptophan to tyrosine; tyrosine to tryptophan or phenylalanine; and valine to isoleucine or leucine.

In addition to a deletion or substitution, a modified protein may possess an insertion of residues, which typically involves the addition of at least one residue in the polypeptide. This may include the insertion of a targeting peptide or polypeptide or simply a single residue. Terminal additions, called fusion proteins, are discussed below.

The term “biologically functional equivalent” is well understood in the art and is further defined in detail herein. Accordingly, sequences that have between about 70% and about 80%, or between about 81% and about 90%, or even between about 91% and about 99% of amino acids that are identical or functionally equivalent to the amino acids of a control polypeptide are included, provided the biological activity of the protein is maintained. A recombinant protein may be biologically functionally equivalent to its native counterpart in certain aspects.

It also will be understood that amino acid and nucleic acid sequences may include additional residues, such as additional N- or C-terminal amino acids or 5′ or 3′ sequences, and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region or may include various internal sequences, i.e., introns, which are known to occur within genes.

As used herein, a protein or peptide generally refers, but is not limited to, a protein of greater than about 200 amino acids, up to a full length sequence translated from a gene; a polypeptide of greater than about 100 amino acids; and/or a peptide of from about 3 to about 100 amino acids. For convenience, the terms “protein,” “polypeptide,” and “peptide are used interchangeably herein.

As used herein, an “amino acid residue” refers to any naturally occurring amino acid, any amino acid derivative, or any amino acid mimic known in the art. In certain embodiments, the residues of the protein or peptide are sequential, without any non-amino acids interrupting the sequence of amino acid residues. In other embodiments, the sequence may comprise one or more non-amino acid moieties. In particular embodiments, the sequence of residues of the protein or peptide may be interrupted by one or more non-amino acid moieties.

Accordingly, the term “protein or peptide” encompasses amino acid sequences comprising at least one of the 20 common amino acids found in naturally occurring proteins, or at least one modified or unusual amino acid.

Certain embodiments of the present invention concern fusion proteins. These molecules may have a therapeutic protein linked at the N- or C-terminus to a heterologous domain. For example, fusions may also employ leader sequences from other species to permit the recombinant expression of a protein in a heterologous host. Another useful fusion includes the addition of a protein affinity tag, such as a serum albumin affinity tag or six histidine residues, or an immunologically active domain, such as an antibody epitope, preferably cleavable, to facilitate purification of the fusion protein. Non-limiting affinity tags include polyhistidine, chitin binding protein (CBP), maltose binding protein (MBP), and glutathione-S-transferase (GST).

Methods of generating fusion proteins are well known to those of skill in the art. Such proteins can be produced, for example, by de novo synthesis of the complete fusion protein, or by attachment of the DNA sequence encoding the heterologous domain, followed by expression of the intact fusion protein.

Production of fusion proteins that recover the functional activities of the parent proteins may be facilitated by connecting genes with a bridging DNA segment encoding a peptide linker that is spliced between the polypeptides connected in tandem. The linker would be of sufficient length to allow proper folding of the resulting fusion protein.

### VIII. KITS AND DIAGNOSTICS

In various aspects of the invention, a kit is envisioned containing the necessary components to purify exosomes from a body fluid or tissue culture medium. In other aspects, a kit is envisioned containing the necessary components to isolate exosomes and transfect them with a CRISPR system. The kit may comprise one or more sealed vials containing any of such components. In some embodiments, the kit may also comprise a suitable container means, which is a container that will not react with components of the kit, such as an eppendorf tube, an assay plate, a syringe, a bottle, or a tube. The container may be made from sterilizable materials such as plastic or glass.

The kit may further include an instruction sheet that outlines the procedural steps of the methods set forth herein, and will follow substantially the same procedures as described herein or are known to those of ordinary skill. The instruction information may be in a computer readable media containing machine-readable instructions that, when executed using a computer, cause the display of a real or virtual procedure of purifying exosomes from a sample and transfecting or electroporating a CRISPR system therein.

### IX. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Materials and Methods

Isolation and purification of exosomes. Exosomes were purified by differential centrifugation processes, as described previously (Alvarez-Erviti et al., 2011; El-Andaloussi et al., 2012). Supernatant was collected from cells that were cultured in media containing exosomes-depleted FBS for 48 hours, and was subsequently subjected to sequential centrifugation steps for 800 g for 5 minutes, and 2000 g for 10 minutes. This resulting supernatant was then filtered using 0.2 μm filters in culture bottles, and a pellet was recovered at 28,000 g in a SW 32 Ti rotor after 2 hours of ultracentrifugation (Beckman). The supernatant was aspirated and the pellet was resuspended in PBS and subsequently ultracentrifuged for another 2 hours. The purified exosomes were then analyzed and used for experimental procedures.

Electroporation of Exosomes and Liposomes.

1×10⁸-3×10⁸ exosomes (measured by nanosight analysis) and the indicated amount of RNA were mixed in 400 μl of electroporation buffer (1.15 mM potassium phosphate, pH 7.2, 25 mM potassium chloride, 21% Optiprep™). Exosomes were electroporated using a 4 mm cuvette using a Gene Pulser Xcell™ Electroporation System (BioRad) as previously described (Alvarez-Erviti et al., 2011; El-Andaloussi et al., 2012). After electroporation, exosomes were treated with protease-free RNAse A (Sigma Aldrich) followed by addition of 10× concentrated RNase inhibitor (Ambion), and washed with PBS under ultracentrifugation methods, as described above.

Exosome Transfection.

For in vitro transfection using exosomes, exosomes were electroporated and washed with PBS as described above, and 200,000 cells in a 6-well plate were treated with exosomes for the required time as described for each assay and subsequently washed with PBS and used for further analysis.

Real-Time PCR Analyses.

RNA was retro-transcribed with MultiScribe Reverse Transcriptase (Applied Biosystems) and oligo-d(T) primers following total RNA purification with TRIzol® (Invitrogen), according to the manufacturer's directions. Real-time PCR analyses were performed on an ABI PRISM® 7300HT Sequence Detection System Instrument using SYBR® Green Master Mix (Applied Biosystems). The transcripts of interest were normalized to 18S transcript levels. Each measurement was performed in triplicate. Threshold cycle, the fractional cycle number at which the amount of amplified target reached a fixed threshold, was determined and expression was measured using the 2^{-ΔCct} formula.

Western Blot.

To deduce the protein expression of cells after treatment with exosomes after 24 hours, cells were harvested in RIPA buffer and protein lysates were normalized using Bradford quantification. 40 μg of lysates were loaded onto acrylamide gels for electrophoretic separation of proteins under denaturing conditions and transferred onto PVDF membranes (ImmobilonP) by wet electrophoretic transfer. The membranes were then blocked for 1 hour at room temperature with 5% non-fat dry milk in PBS/0.05% Tween-20 and incubated overnight at 4° C. with the appropriate primary antibodies. Secondary antibodies were incubated for 1 hour at room temperature. Washes after antibody incubations were done on an orbital shaker, three times at 15 min intervals, with 1×PBS 0.05% Tween®-20. Membranes were developed with chemiluminescent reagents from Pierce, according to the manufacturer's directions and chemiluminescence captured on film.

Transfection and Validation of CRISPR-Cas9-sgRab27a-2 Cells.

HEK293T cells were transfected with CRISPR-Cas9 vector control or CRISPR-Cas9-sgRab27a-2 by treatment with lipofectamine for 72 h. Cells were then selected with 1 μg/ml puromycin for 10 days to obtain stable HEK293T CRISPR-Cas9 vector control and CRISPR-Cas9-sgRab27a-2 cells. The stable cells were then cultured with 1 μg/ml puromycin containing selection medium. DNA and RNA were extracted from the stable cell lines as described above and the Cas9 levels were determined using qPCR and RT-qPCR.

Exosome Collection and Validation.

Exosomes were collected from non-transfected HEK293T cells, as well as stable HEK293T CRISPR-Cas9 vector control and CRISPR-Cas9-sgRab27a-2 cells, as described above. The quality of exosomes was validated by Nanosight.

CRISPR-Cas9 Genome Editing.

To ensure the presence, and determine the quantities of the appropriate vectors, exosomal DNA and RNA were extracted, and qPCR and RT-qPCR were performed to detect Cas9 vector control levels in exosomes, as well as the levels of the sgRNA against Rab27a-2. Further, Cas9 protein levels were assessed in both cells and exosomes by Western blot, using either anti-Flag antibody or Cas9 antibody, with Vinculin or CD9 as controls, respectively. The T7/SURVEYOR assay was used to determine whether DNA editing had occurred in both cells and exosomes.

Treatment of BxPC-3 Adenocarcinoma Cells with Exosomes.

3×10¹⁰ exosomes collected from HEK293T blank cells, HEK293T CRISPR-Cas9 vector control and CRISPR-Cas9-sgRab27a-2 stable cells were treated into BxPC-3 adenocarcinoma cells every 24 h as described above, either once or twice. DNA and RNA were extracted from the recipient cells, and Cas9 levels or sgRNA levels were detected from both the DNA and RNA using qPCR and RT-qPCR. The T7/SURVEYOR assay was then used to determine editing in the recipient BxPC-3 cells.

Treatment of BJ Cells with CRISPR-Cas9 Exosomes Isolated from BJ cells.

Exosomes were collected from BJ cells, as above. Nanosight was used to validate the exosomes. Exosome markers CD9, CD81, Flotillin and TSG101 were detected by Western blot to further confirm the exosomes. 1×10¹⁰ isolated and validated BJ cell exosomes were electroporated with 15 μg CRISPR-Cas9-GFP plasmid, and then treated with or without DNase. Following DNase treatment, exosomal DNA was extracted and Cas9 levels were evaluated by qPCR. Copy number was further calculated by absolute qPCR with CRISPR-Cas9-GFP plasmid as a standard. The electroporated exosomes with DNase were then transfected into BJ cells for 24 h as described above. Cas9 levels were then detected from both DNA and mRNA using qPCR or RT-qPCR.

Transduction of BxPC-3 Cells with HEK293T/CRISPR-Cas9 Media.

HEK293T cells were transfected using packaging plasmids together with CRISPR-Cas9 Rab27b-1/2, or empty control plasmids, by lipofectamine 2000 as above. The medium containing lentivirus was harvested and then transduced into BxPC-3 cells. The transduced cells were further selected with 0.4 μg/mL puromycin, and single clones of BxPC-3/CRISPR-Cas9-sgRab27b cells were picked, clonally expanded, and validated by both Western blot and the T7/SURVEYOR assay. Rab27b and Rab27a protein levels were then evaluated in all the single clones. The T7/SURVEYOR assay was also used to validate that gene editing had occurred in all the clones. BxPC-3/CRISPR-Cas9 vector control stable cells and single clones BxPC-3/CRISPR-Cas9-sgRab27b-1 C3, BxPC-3/CRISPR-Cas9-sgRab27b-2 C6 were cultured with 0.4 μg/ml puromycin containing selection medium. Exosomes were collected from the abovementioned cells, as were secreted exosomes, followed by Nanosight validation. Exosomal DNA and RNA were extracted, and qPCR was performed to detect Cas9 levels in exosomes, as well as RT-qPCR to detect sgRNA against Rab27b-1/2. Cas9 and Rab27b protein levels were assessed in both cells and exosomes by Western blot, and the T7/SURVEYOR assay was used to determine whether DNA editing had occurred in both cells and exosomes.

Evaluation of Protein Concentration from Exosomes.

BxPC-3/CRISPR-Cas9 vector control stable cells and single clones BxPC-3/CRISPR-Cas9-sgRab27b-1 Clone 3 (C3), BxPC-3/CRISPR-Cas9-sgRab27b-2 Clone 6 (C6) were cultured, and exosomes were collected as described above. The exosomes collected from BxPC-3/CRISPR-Cas9 vector control stable cells and single clones BxPC-3/CRISPR-Cas9-sgRab27b-1 C3, BxPC-3/CRISPR-Cas9-sgRab27b-2 C6 were lysed and protein content was assessed by BCA kit according to the manufacturer's instructions.

Cellular Proliferation Assays.

To insure cellular proliferation was unaffected by the presence of CRISPR-Cas9 or gene editing, controls and CRISPR-Cas9 treated cells were evaluated. 100 μL of BxPC-3 cells without treatment, BxPC-3 with CRISPR-Cas9 empty vector control, BxPC-3/CRISPR-Cas9-sgRab27b-1 C3 and BxPC-3/CRISPR-Cas9-sgRab27b-2 C6 cells were seeded in 96-well plates at the concentration of 1×10⁵ cells/mL. Cell proliferation was evaluated using a MTT assay at different time points.

In Vitro Transcription of sgRab27b.

To generate in vitro transcribed sgRab27b, sgRab27b-1/2 was first amplified by PCR, and then the PCR products were purified using the Qiagen® PCR purification kit. The purified PCR products of sgRab27-1/2 were in vitro transcribed using the MEGAshortscript™ kit (Thermo Fisher Scientific® Cat. No. 1354) according to the manufacturer's instructions. The RNA quality was further evaluated by electrophoresis using an 8M urea polyacrylamide gel. To generate in vitro transcribed Cas9, Cas9 was amplified by PCR, with the PCR products further purified using the Qiagen® PCR purification kit. Purified Cas9 PCR products were in vitro transcribed using the mMESSAGE mMACHINE® T7 Ultra Kit. Formaldehyde gels were used to detect Cas9 RNA quality.

Treatment of Cells with In Vitro Transcribed RNA.

To evaluate transfection and CRISPR-Cas9 efficiency HEK293T/CRISPR-Cas9 vector control cells were transfected with 1 μg IVT-sgRab27b RNA using either lipofectamine 2000, Exo-Fect/exosome transfection reagent, or electroporated exosomes for 72 h. Following transfection, DNA was extracted, and T7/SURVEYOR assay was performed to determine whether gene editing had occurred. HEK293T cells and BxPC-3 cells were transfected with Cas9 mRNA using lipofectamine 2000, Exo-Fect/exosome transfection reagent, or treated with 1×10⁹ MSC exosomes electroporated with Cas9 mRNA for 48 h. Western blotting was performed to detect Cas9 protein level.

Evaluation of Exo-Fect/Exosome Treatment.

Hekt293T cells were treated with 10 μg plasmids (CRISPR-Cas9-lenti-V2 vector control, CRISPR-Cas9-lenti-V2-sgRab27b-1, CRISPR-Cas9-GFP vector control) using Exo-Fect/exosome transfection reagent every 24 h for 4 times (day 1, 2, 3, 4). CRISPR-Cas9-GFP cells were imaged on day 5 to detect GFP expression. Cells were also collected for nucleic acid and protein isolation on day 5. DNA, RNA and protein were extracted. Relative Cas9 expression levels and 1/Ct values were determined by qPCR for cells transfected with each plasmid and Western blots were used to detect Cas9 protein levels. A T7/SURVEYOR assay was performed to determine the occurrence of gene editing in HEK293T cells after treatment with CRISPR-Cas9-lenti-V2-sgRab27b-1 plasmid. The same experiment was repeated with BxPC-3 cells.

sgmKras Editing of KPC689 Cells.

KPC689 cells were transfected with μg of control plasmids, or with a CRISPR-Cas9-sgmKras^{G12D}-lenti-V2 plasmid by lipofectamine 2000 for 48 h. Following transfection, CRISPR-Cas9-GFP vector control cells were imaged to determine transfection efficiency. DNA, RNA and protein were extracted from all cultures, as above. Relative Cas9 and mKras^{G12D} expression levels were determined by qPCR, and as above, a T7/SURVEYOR assay was performed to check whether gene editing had occurred in KPC689 cells after transfection by lipofectamine Fresh KPC689 cells were treated with 10 μg plasmids of CRISPR-Cas9-sgmKras^{G12D} with GFP backbone, or its vector control using Exo-Fect/exosome transfection reagent every 24 h for 3 days. Cells were imaged for GFP expression to determine transfection efficiency, and were collected on day 4. DNA, RNA and protein were extracted, and relative Cas9 and mKRas^{G12D} expression levels were determined by qPCR. A T7/SURVEYOR assay was performed to confirm gene editing in KPC689 cells following treatment with CRISPR-Cas9-GFP-mKras^{G12D} plasmids.

Transfection and Validation of Doxycycline Inducible CRISPR-Cas9 Plasmids.

HEK293T cells were transfected with a mixture of lentiviral packaging plasmids together with CRISPR-Cas9 doxycycline inducible plasmids by lipofectamine 2000. The medium containing lentivirus was harvested and then transduced into Panc1 cells. The transduced cells were further selected with 1 μg/ml puromycin. The stable Panc1 cells with inducible Cas9 were maintained by culturing with 1 μg/ml doxycycline. Exosomes were collected from Panc1 inducible cells treated with, or without, doxycycline. Western blotting was used to check Cas9 protein level in both cells and exosomes. The Panc1 inducible cells were treated with 2 μg IVT-sgRNA against hKras^{G12D}, 1 μg hKras^{G12D} plasmid by lipofectamine, Fugene or Exo-Fect for 72 h. T7/SURVEYOR assays were then performed to confirm gene editing in Panc1 inducible cells.

Treatment of Panc1 Cell Lines with with CRISPR-Cas9-sghKRasG^{12D}.

Panc1-Cas9 and Panc1 sghKras^{G12D} T1 stable cell lines were established using a lentivirus based method. Expression of Cas9 protein in Panc1-Cas9 cells was confirmed by Western blot. Panc1 cells which had not been transfected were treated with CRISPR-Cas9-sghKras^{G12D} in either lenti-V2, GFP, or puromycin backbones using lipofectamine, Exo-Fect or electroporated exosomes. Panc1-Cas9 stable cells were treated with sghKras^{G12D} plasmids using lipofectamine, Exo-Fect or electroporated exosomes, and Panc1 sghKrasG¹²D T1 stable cells were transfected with 10 μg or 20 μg of Cas9 plasmids with either GFP or puromycin backbones for 24 h. T7/SURVEYOR assay was performed to confirm that gene editing had occurred.

Treatment of Implanted KPC689 Tumors In Vivo.

KPC689 cells were implanted subcutaneously into the back of each mouse. The mice were divided into 4 groups, with 1 or 2 mice per group. Group 1 was treated with 1×10⁹ exosomes and 10 μL Exo-Fect. Group 2 was treated with 10 μg Cas9-GFP-sgmKras^{G12D}-mK1 plasmid. Group 3 was treated with 1×10⁹ exosomes, 10 μg Cas9-GFP-vector control plasmid and 10 μL Exo-Fect. Group 4 was treated with 1×10⁹ exosomes, 10 μg Cas9-GFP-sgmKras^{G12D}-mK1 plasmid and 10 μL Exo-Fect. Mice in each group were injected intravenously (I.V.) and intratumorally (I.T.) every day for two weeks. Tumor length (a, mm) and width (b, mm) as well as body weight were measured and tumor volume was calculated.

### Example 2—Establishment of CRISPR-Cas9 Exosomes

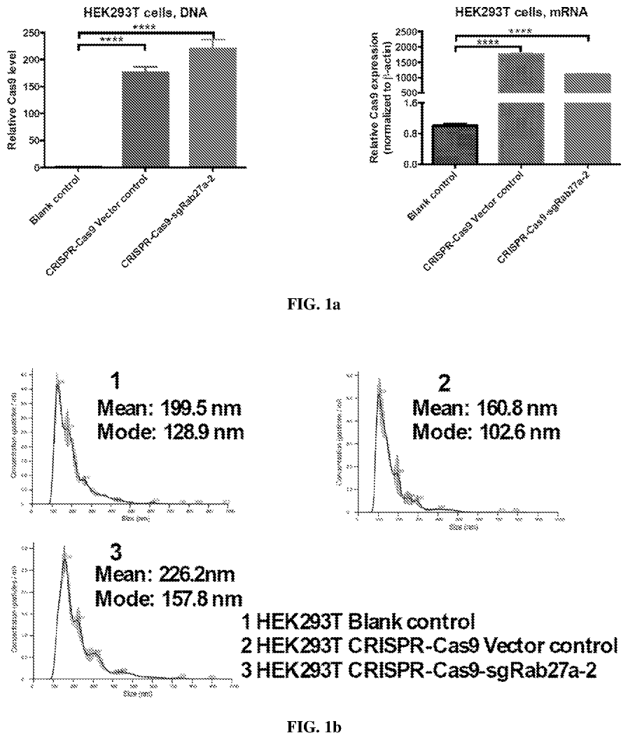

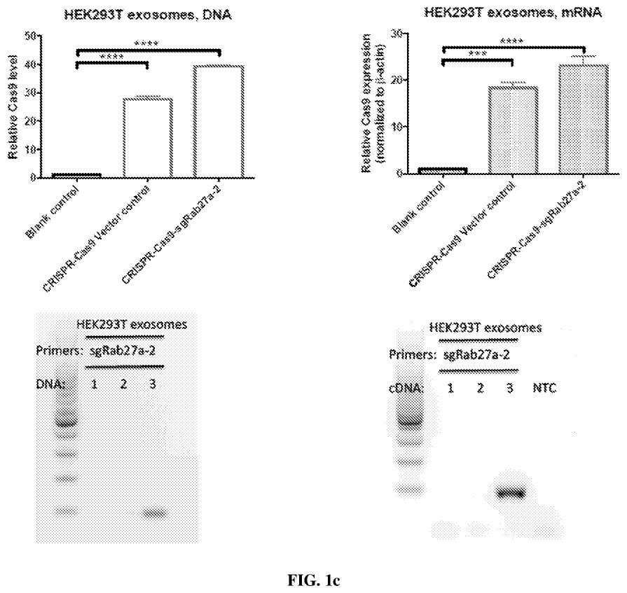

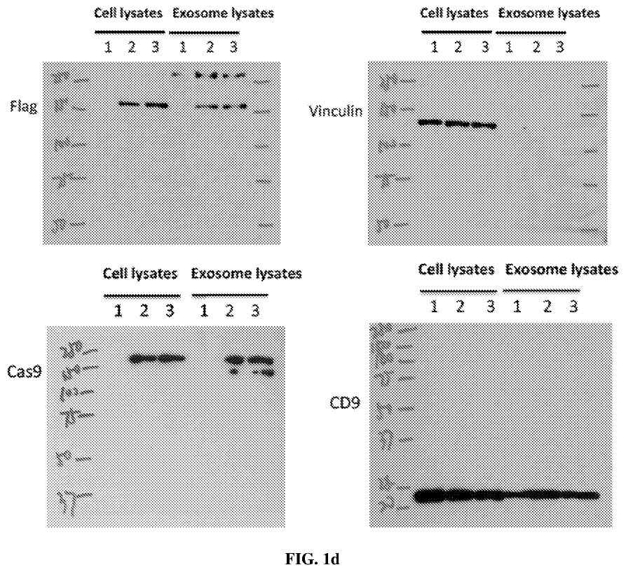

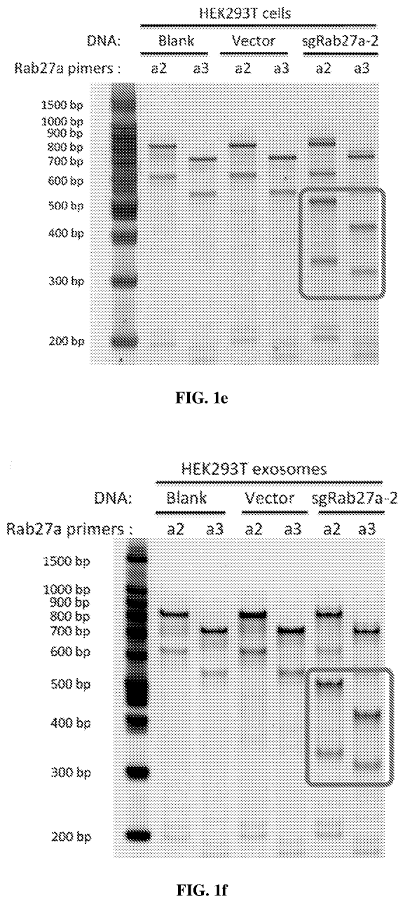

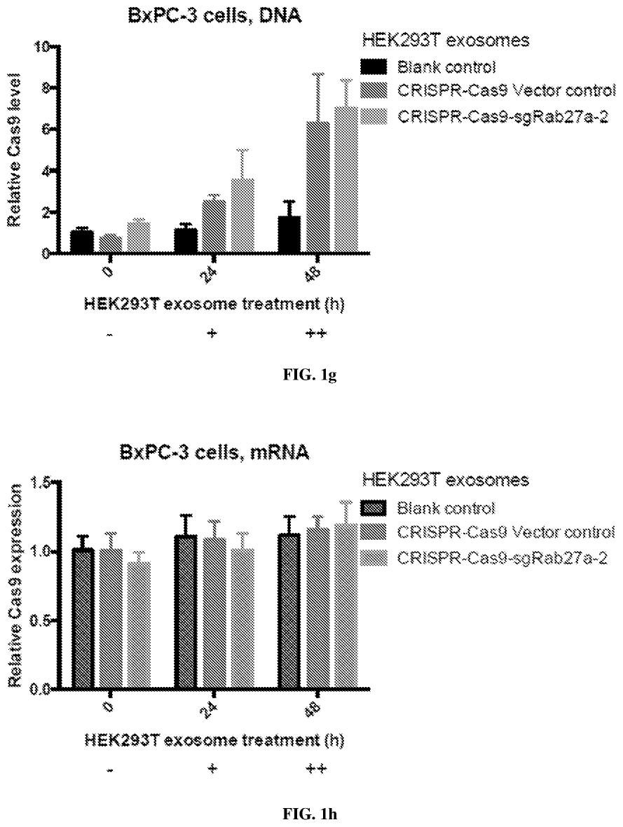

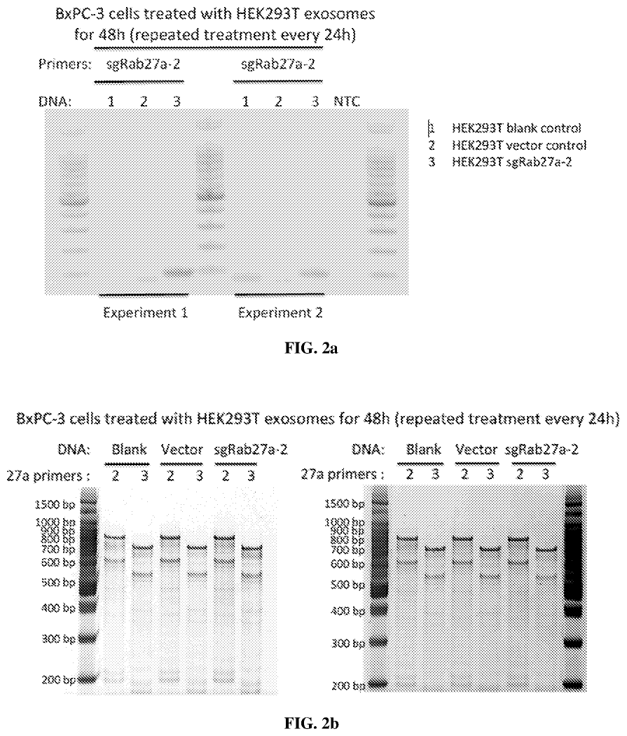

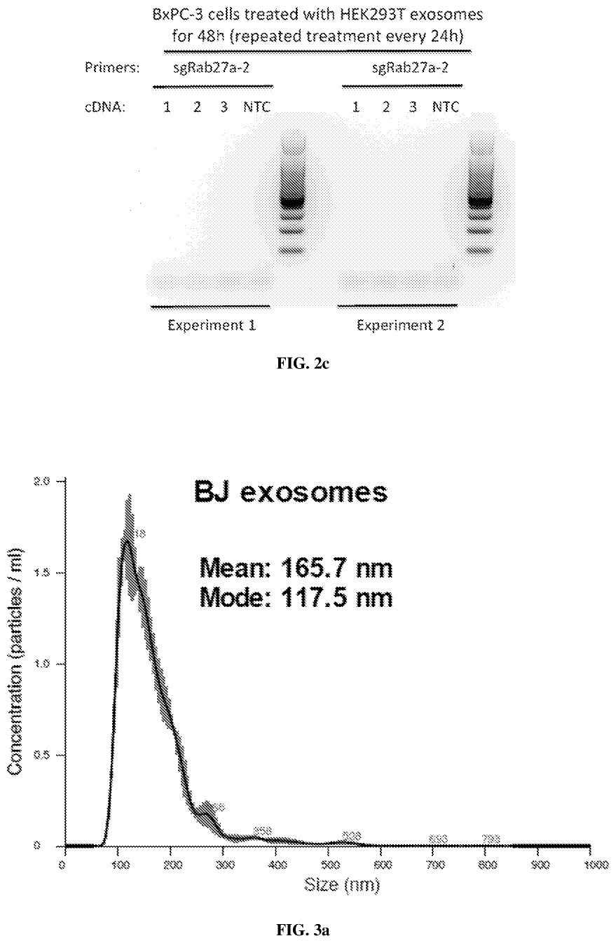

DNA and RNA were extracted from HEK293T transfected with CRISPR-Cas9 vector control and CRISPR-Cas9-sgRab27a-2, and Cas9 levels were determined using quantitative real-time PCR (qPCR) (FIG. 1a). Both vectors were transfected efficiently, and transfected cells showed significantly greater Cas9 expression, relative to a (3-actin control. Exosomes were collected from HEK293T blank cells, as well as stable HEK293T CRISPR-Cas9 vector control and CRISPR-Cas9-sgRab27a-2 cells. Nanosight validation of the exosomes can be seen in FIG. 1b. Exosomal DNA and RNA were extracted, and qPCR was performed to detect Cas9 levels in exosomes, as well as sgRNA against Rab27a-2. Similarly to the cells, both vector control and vector with guide RNA were expressed in the exosomes (FIG. 1c). To confirm Cas9 expression, Cas9 protein levels were assessed in both cells and exosomes by Western blot, using either anti-Flag antibody or Cas9 antibody, with Vinculin or CD9 as controls, respectively (FIG. 1d). The T7/SURVEYOR assay was used to confirm DNA editing in both cells and exosomes, and is visible within the boxed in areas of FIGS. 1e and 1f. Exosome treated BxPC-3 cells were evaluated for the presence of Cas9 DNA and Cas9 expression (FIGS. 1g and 1h). Cells treated twice had an increase in Cas9 DNA, as can be seen in FIG. 1g. Exosome treated BxPC-3 cells were tested for the presence of the guide RNA to confirm its presence (FIGS. 2a and 2c), and while the DNA was apparent, there was no RNA expression. This was confirmed by the lack of activity in the T7/SURVEYOR assay (FIG. 2b).

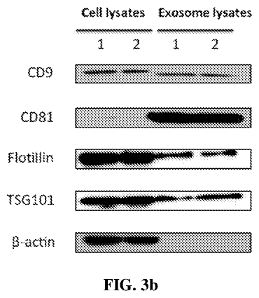

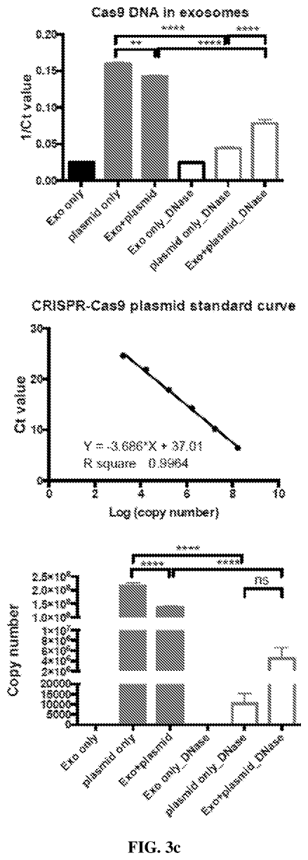

Exosomes were collected from BJ cells, and confirmed by nanosight analysis as depicted in FIG. 3a. Exosome markers CD9, CD81, Flotillin and TSG101 were detected by Western blot to further confirm the exosomes (FIG. 3b). BJ exosomes were electroporated with 15 ug CRISPR-Cas9-GFP plasmid, and then treated with or without DNase. Cas9 DNA was detected strongly in samples that were not treated with DNase, and were detected more efficiently in DNase treated samples which contained both the exosomes and the plasmid than plasmid alone (FIG. 3c). Plasmid copy number was determined using a standard curve generated from the 1/Ct value (FIG. 3c). The electroporated exosomes with DNase were treated into BJ cells for 24 h, and Cas9 levels increased in both DNA and mRNA when compared to blank exosomes (FIG. 3d).

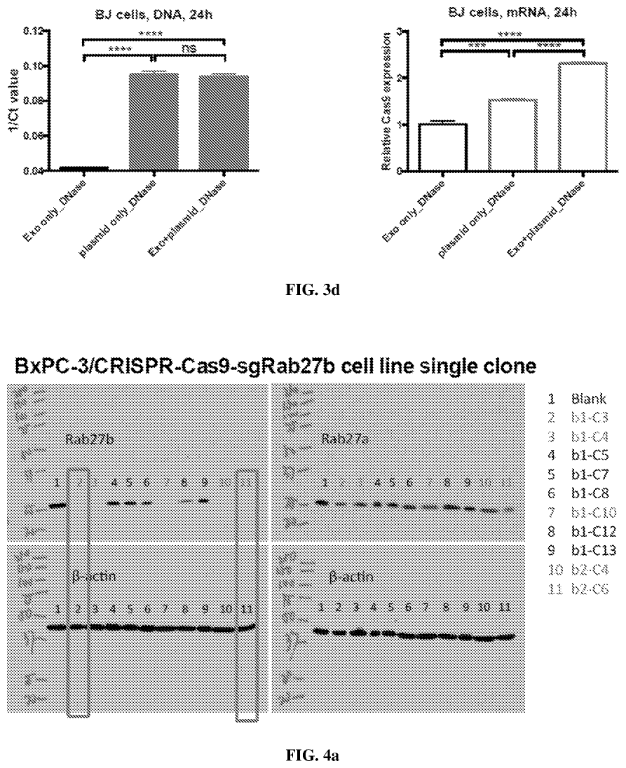

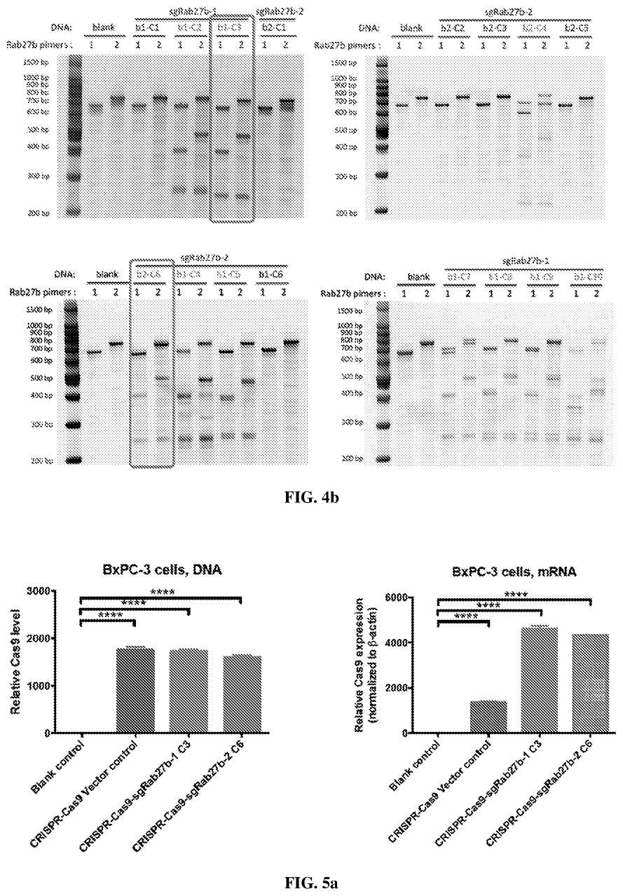

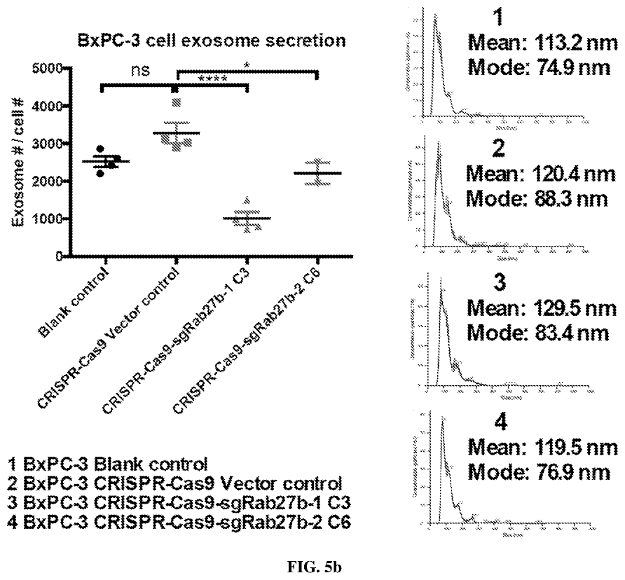

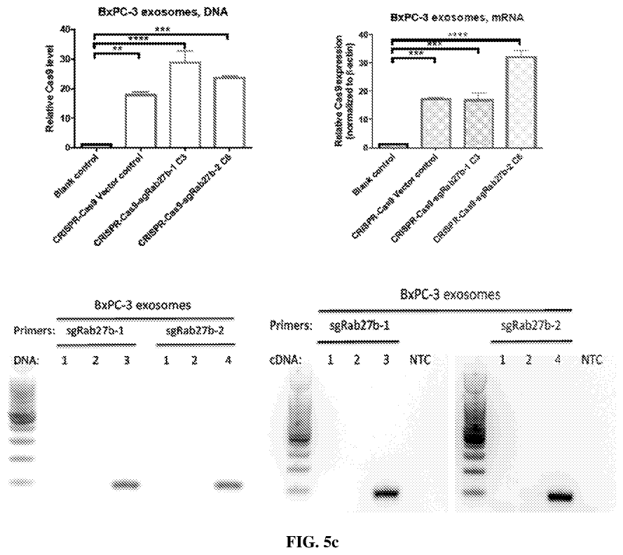

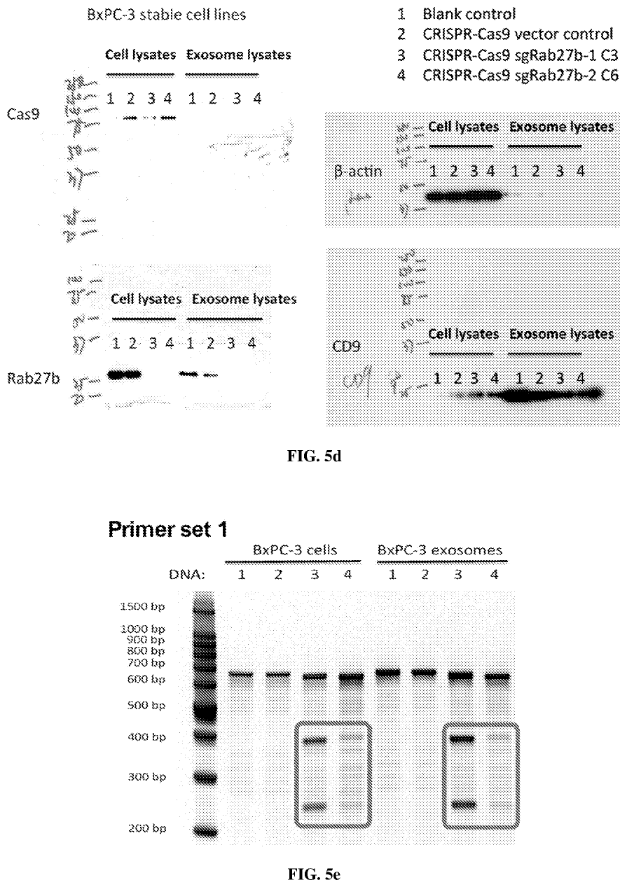

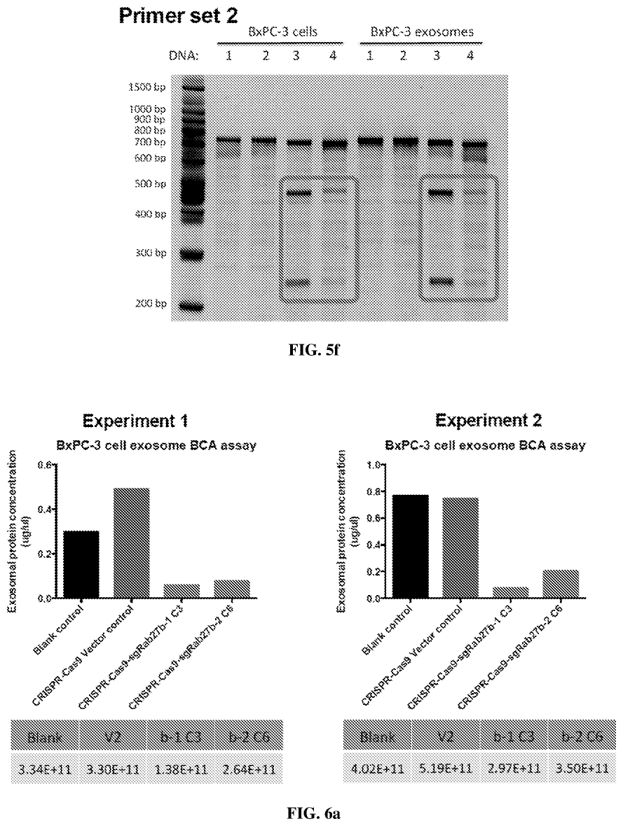

Clonally expanded BxPC-3 cells which had been transduced with lentivirus media containing a CRISPR-CAS9 Rab27b-1/2 plasmid were validated by both Western blot (FIG. 4a) and T7/SURVEYOR assay (FIG. 4b). Two clones were found to be active, and are seen boxed in FIG. 4b. BxPC-3/CRIPSR-Cas9-sgRab27b-1 clone 3 (C3) and BxPC-3/CRISPR-Cas9-sgRab27b-2 clone 6 (C6) were used for further experiments. BxPC-3/CRISPR-Cas9-sgRab27b-1 C3 and BxPC-3/CRISPR-Cas9-sgRab27b-2 C6 were cultured with 0.4 μg/ml puromycin containing selection medium, and the DNA and RNA were extracted. The presence of Cas9 DNA was confirmed by qPCR, while Cas9 expression was confirmed by RT-qPCR, and found to be significantly greater than in vector control cells (FIG. 5a). Exosomes were collected from these cells and confirmed by nanosight analysis (FIG. 5b). Exosomal DNA and RNA were extracted, and qPCR was performed to detect Cas9 levels in exosomes, and it was found that BxPC-3/CRISPR-Cas9-sgRab27b-2 C6 had significantly greater Cas9 expression than BxPC-3/CRISPR-Cas9-sgRab27b-2 C3 (FIG. 5c). This was confirmed by detection of the sgRNA against Rab27b-1/2 (FIG. 5c, bottom). Cas9 and Rab27b protein levels were assessed in both cells and exosomes by Western blot, with b-actin or CD9 as controls, respectively, and it was found that Rab27b was knocked down in cells and exosomes carrying the guide RNAs (FIG. 5d). A T7/SURVEYOR assay was used to confirm DNA editing in both cells and exosomes using two different primer sets (FIGS. Se and 5f), and are visible as the darkened bands in the boxed in area.

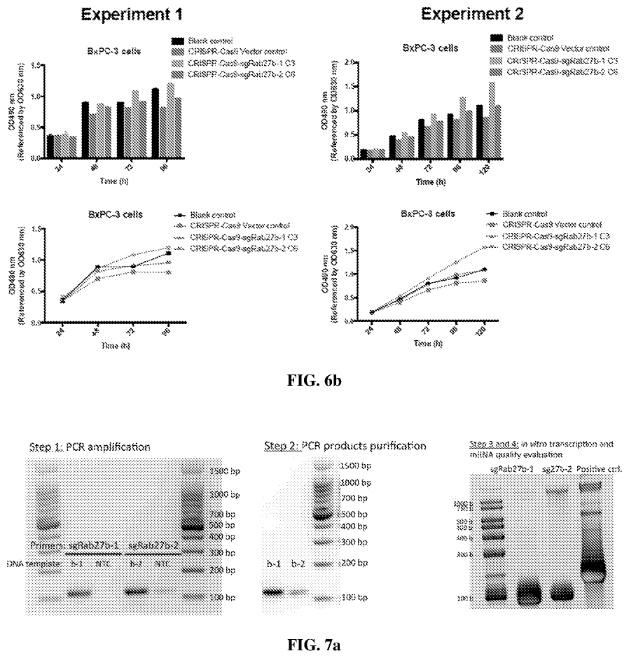

Exosomal protein content of BxPC-3/CRISPR-Cas9 vector control stable cells and clonally expanded BxPC-3/CRISPR-Cas9-sgRab27b-1 C3 amd BxPC-3/CRISPR-Cas9-sgRab27b-2 C6 cells were evaluated by BCA (FIG. 6a). Cellular proliferation was assessed by MTT assay, and the presence of CRISPR-Cas9 had no negative effect on proliferation (FIG. 6b).

Transfection with IVT RNA.

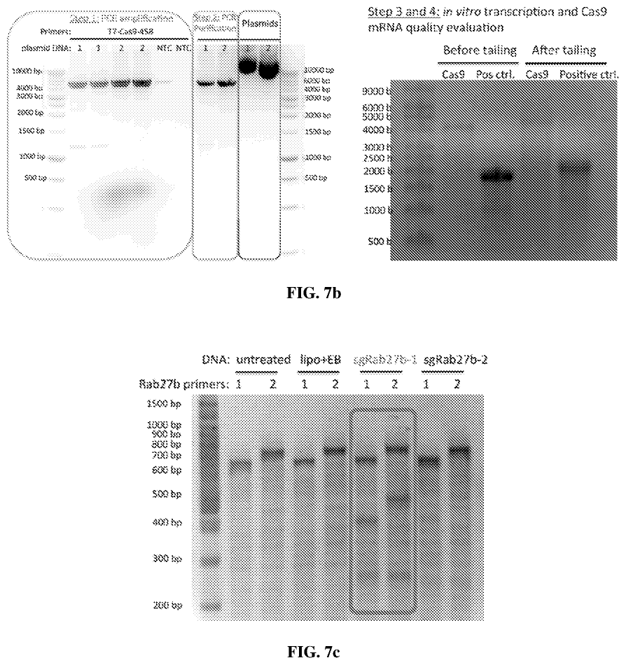

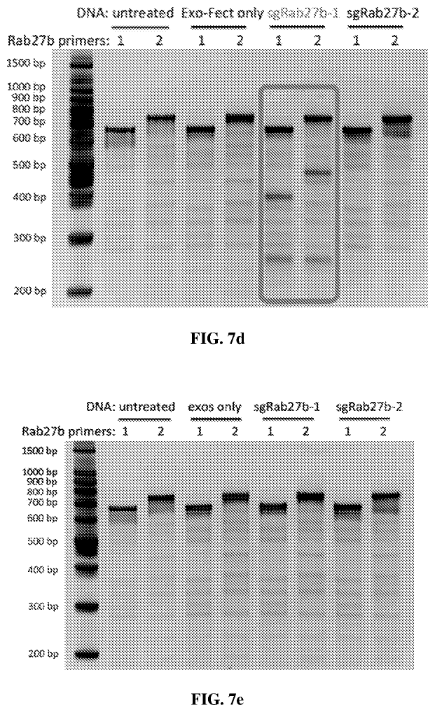

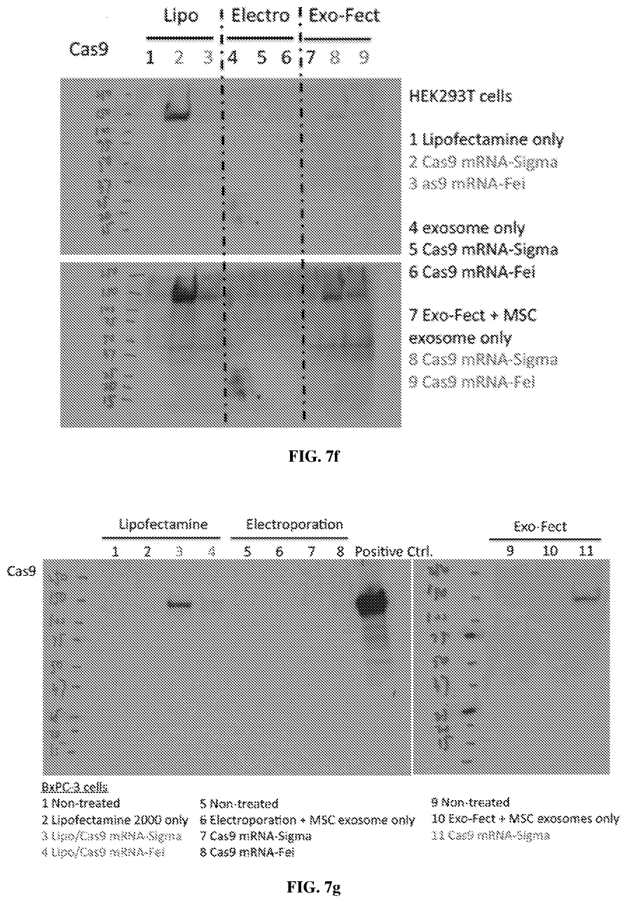

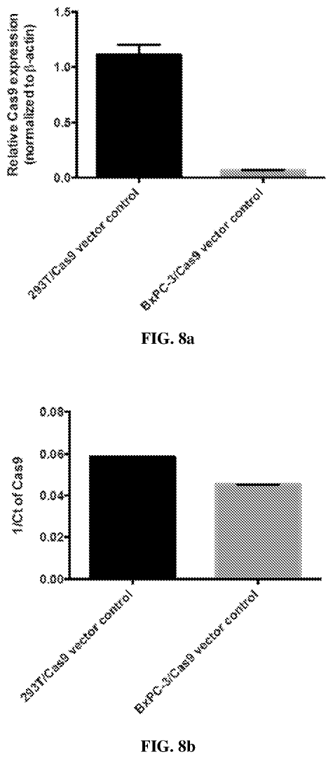

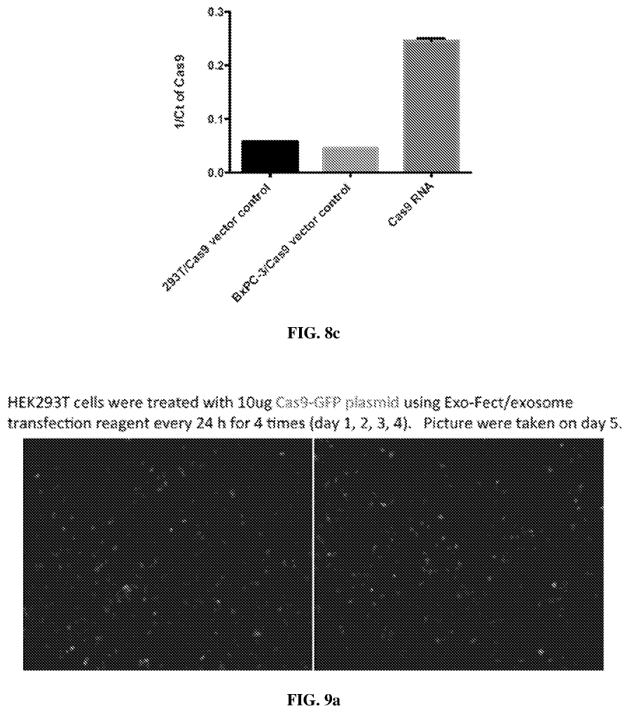

sgRab27b-1/2 was amplified by PCR and purified (FIG. 7a). The purified PCR products of sgRab27-1/2 were then in vitro transcribed as described above and was run on a denaturing gel to resolve the quality (FIG. 7a, right). Cas9 was amplified by PCR and purified (FIG. 7b). Purified Cas9 PCR products were in vitro transcribed and detected by electrophoresis on a formaldehyde gel (FIG. 7b). HEK293T/CRISPR-Cas9 vector control cells were treated with 1 μg IVT-sgRab27b RNA using lipofectamine 2000 (FIG. 7c), Exo-Fect/exosome transfection reagent (FIG. 7d) or electroporated exosomes (FIG. 7e) for 72 h. Gene editing was confirmed in cells treated with RNA and lipofectamine or the exosome transfection reagent (FIGS. 7c and 7d), but not with exosomes. Both HEK293T cells and BxPC-3 cells were transfected with Cas9 mRNA using lipofectamine 2000, Exo-Fect/exosome transfection reagent, or treated with MSC exosomes electroporated with Cas9 mRNA for 48 h, and again only transfection with lipofectamine or exosome transfection reagent yielded cells expressing Cas9 in western blots (FIGS. 7f and 7g). Cas9 controls for both HEK293T cells and BxPC-3 cells with Cas9 vectors are shown in FIGS. 8a-8c.

HEK293T and BxPC-3 Transfection and Gene Editing.

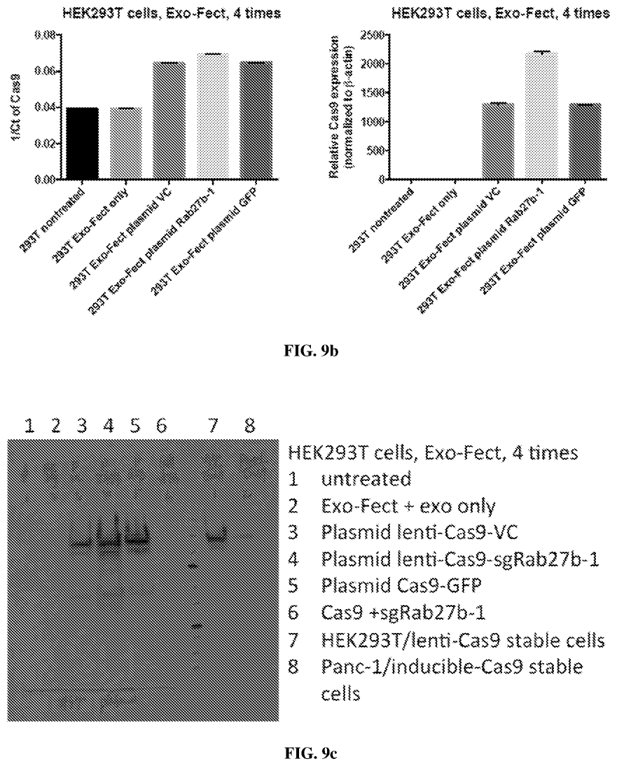

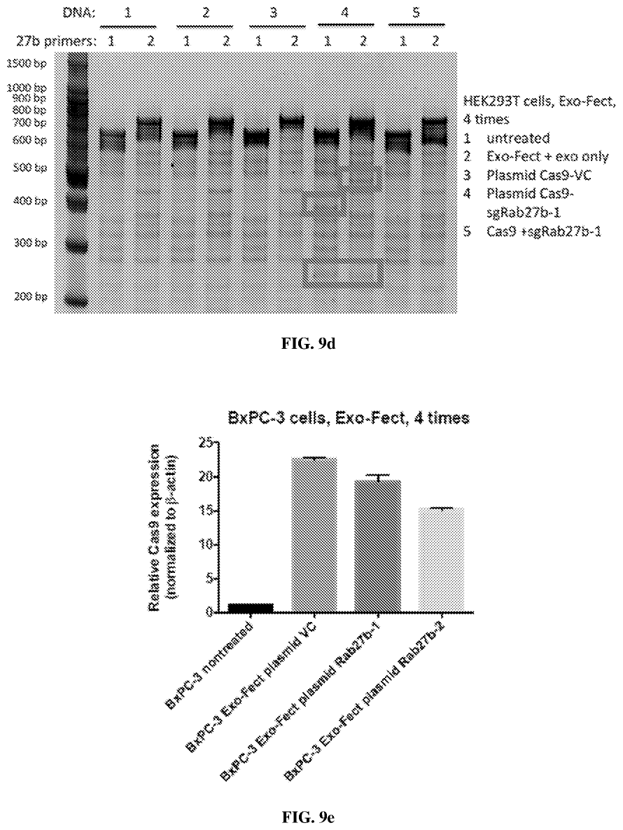

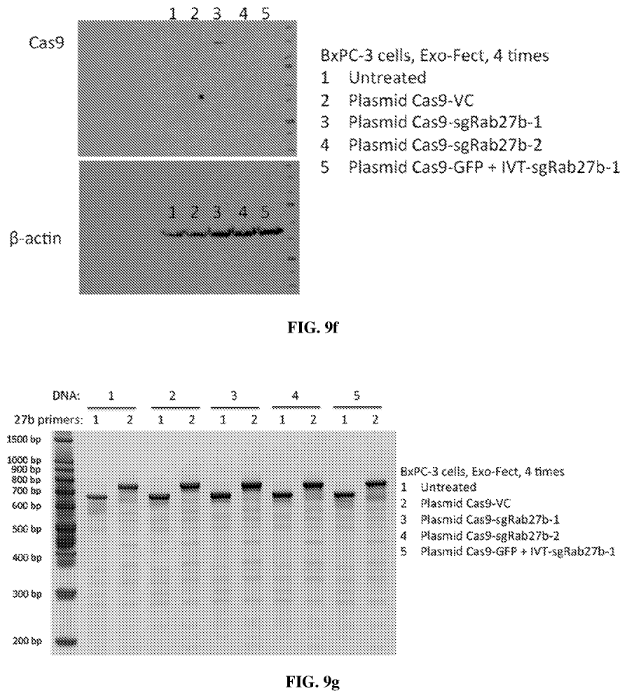

HEK293T cells were treated with 10 μg plasmids (CRISPR-Cas9-lenti-V2 vector control, CRISPR-Cas9-lenti-V2-sgRab27b-1, CRISPR-Cas9-GFP vector control) using Exo-Fect/exosome transfection reagent every 24 h for 4 times (day 1, 2, 3, 4) and transfection efficiency was viewed for the Cas9-GFP transfected cells (FIG. 9a). Relative Cas9 expression level and 1/Ct value were determined by qPCR (FIG. 9b), and western blotting confirmed the presence of Cas9 (FIG. 9C). Gene editing was confirmed with the T7/SURVEYOR assay (FIG. 9d). The same experiments were performed in BxPC-3 cells, though there was no gene editing detectable in the T7/SURVEYOR assay (FIGS. 9e-9g).

KPC689 Transfection and Gene Editing.

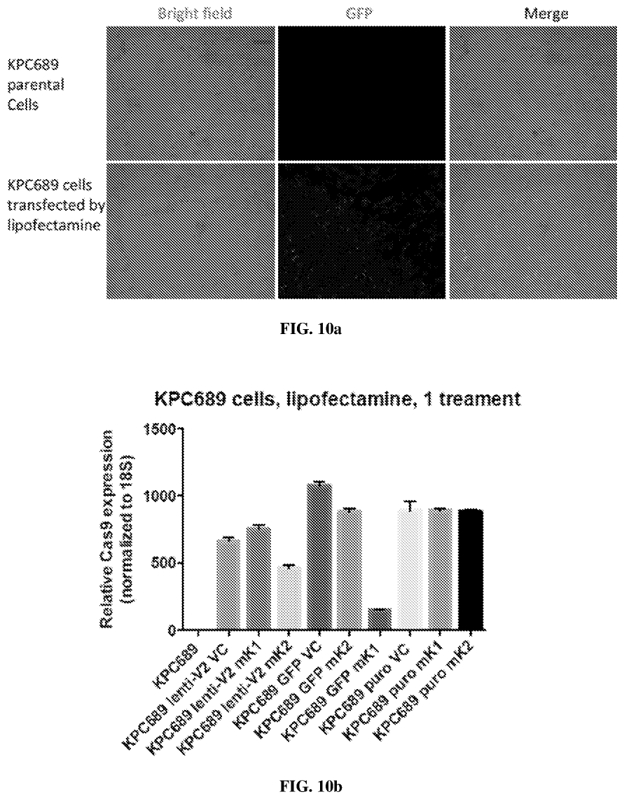

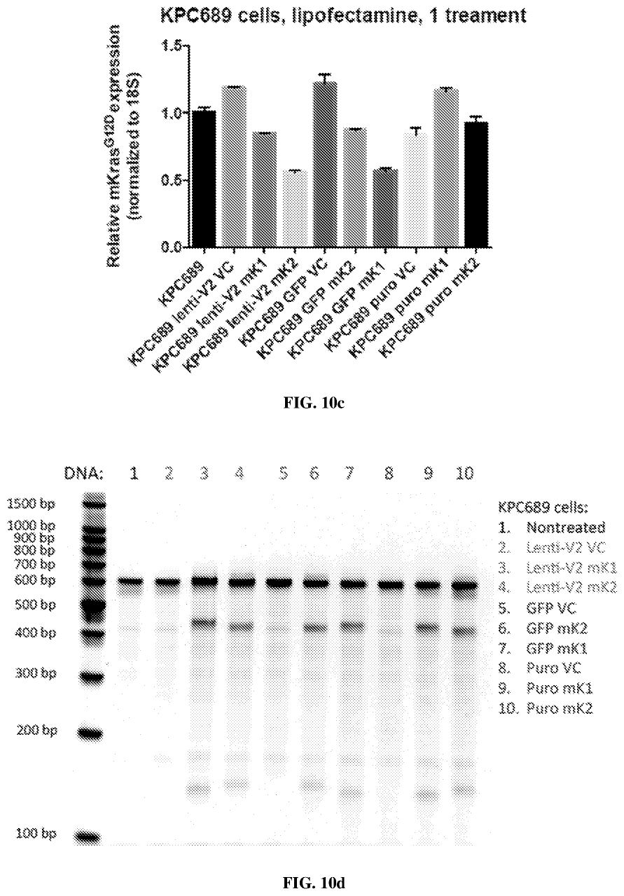

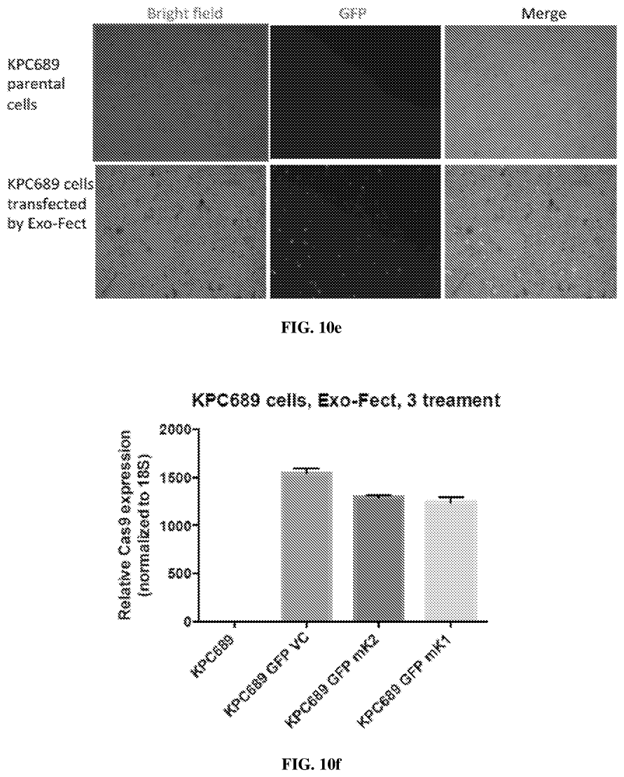

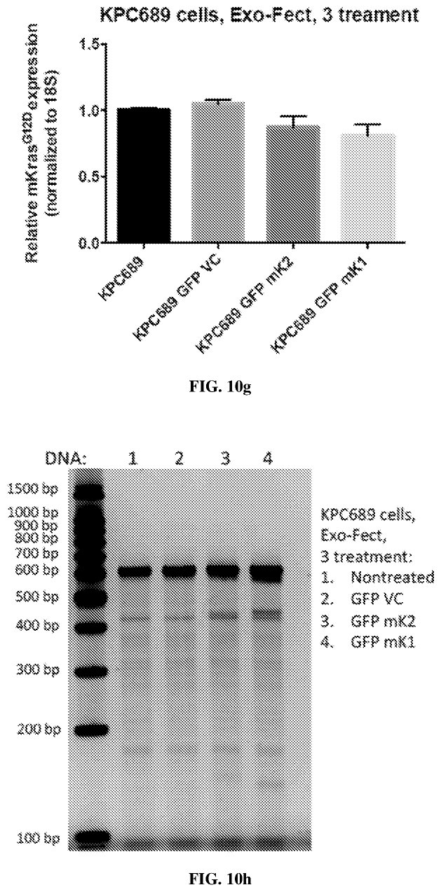

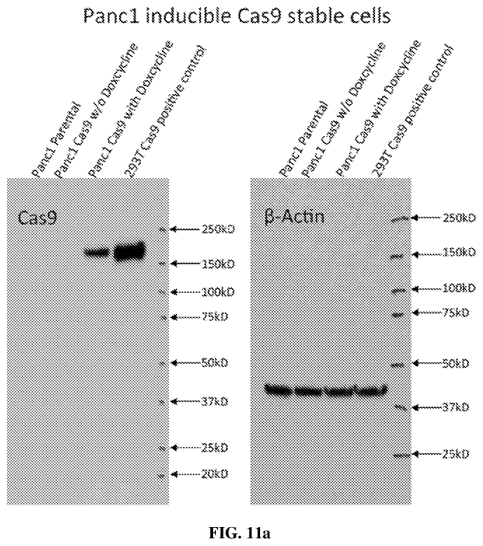

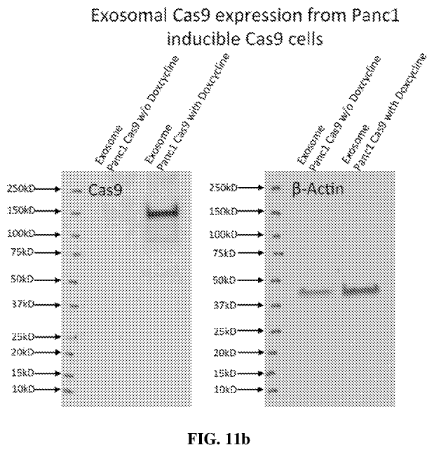

KPC689 cells were transfected with 5 μg plasmids (CRISPR-Cas9-sgmKras^{G12D} with lenti-V2, GFP, puromycin backbone, and the vector controls) by lipofectamine 2000 for 48 h, and transfection was confirmed by imaging cells in the GFP backbone (FIG. 10a). Relative Cas9 level (FIG. 10b) and mKras^{G12D} level (FIG. 10c) were determined by qPCR, and a T7/SURVEYOR assay was performed to check gene editing in KPC689 cells, though it was absent (FIG. 10d) Similar to the previous, KPC689 cells were treated with 10 μg plasmids (CRISPR-Cas9-sgmKras^{G12D} with GFP backbone, and its vector control) using Exo-Fect/exosome transfection reagent and the GFP transfected cells were imaged to confirm the transfection efficiency of Exo-Fect/exosome transfection reagent (FIG. 10e). Relative Cas9 expression level (FIG. 10f) and mKras^{G12D} level (FIG. 10g) were determined by qPCR. A T7/SURVEYOR assay was performed to check gene editing in KPC689 cells after treatment with CRISPR-Cas9-GFP-mKras^{G12D} plasmids, though editing was absent (FIG. 10h).

Treatment with Inducible Plasmids in Exosome Containing Cells.

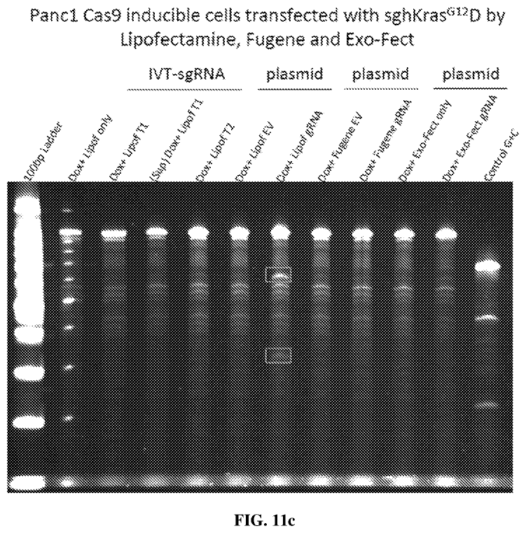

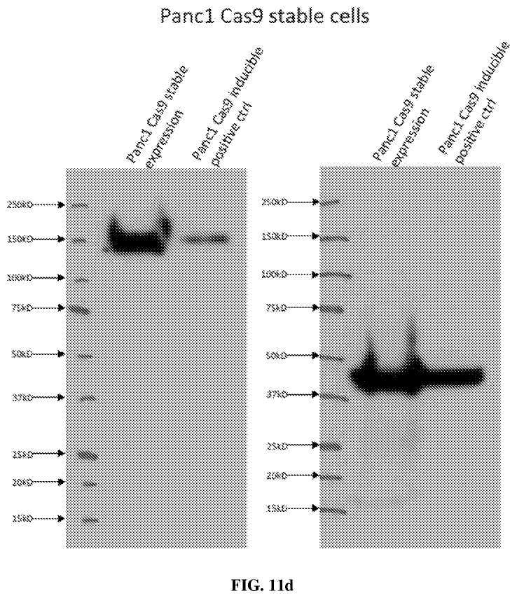

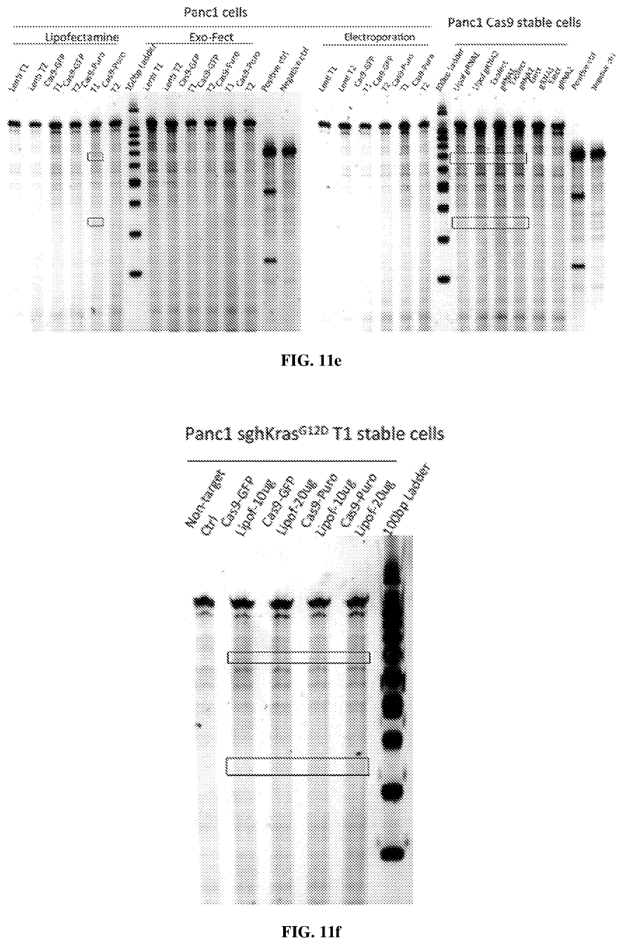

HEK293T cells were transfected using packaging plasmids together with CRISPR-Cas9 doxycycline inducible plasmid by lipofectamine 2000. The medium containing lentivirus was harvested and then transduced into Panc1 cells. The transduced cells were selected with puromycin, and maintained with doxycycline. Exosomes were collected from Panc1 inducible cells treated with or without doxycycline and western blotting was used to confirm Cas9 protein level in cells and exosomes (FIGS. 11a and 11b). The Panc1 inducible cells were treated with 2 μg IVT-sgRNA against hKras^{G12D}, 1 μg hKras^{G12D} plasmid by lipofectamine, Fugene or Exo-Fect for 72 h and a T7/SURVEYOR assay was performed to check gene editing in Panc1 inducible cells, with editing only detected in cells transfected in lipofectamine with a plasmid of the guide RNA (FIG. 11c). Cas9 protein level was determined in Pacn1 Cas9 stable cells by Western blot (FIG. 11d). Panc1 cells were treated with CRISPR-Cas9-sghKras^{G12D} with lenti-V2, GFP, puromycin backbones using lipofectamine, Exo-Fect or electroporated exosomes, while Panc1 Cas9 stable cells were treated with sghKras^{G12D} plasmids using lipofectamine, Exo-Fect or electroporated exosomes as shown, and a T7/SURVEYOR assay was performed to check gene editing in Panc1 cells and Panc1 Cas9 stable cells (FIG. 11e). Gene editing was found in Panc1 cells transformed with Cas9 in a puromycin backbone as seen by the boxed areas, as well as the Panc1-Cas9 stable cell line transfected with guide RNA using either the lipofectamine or Exo-Fect (FIG. 11e). Panc1 sghKras^{G12D} T1 stable cells were established using lentivirus based method. The Panc1 sghKras^{G12D} T1 stable cells were transfected with 10 μg or 20 μg Cas9 plasmids with either GFP or puromycin backbone for 24 h, and a T7/SURVEYOR assay was performed and found gene editing in Panc1 sghKras^{G12D} T1 stable cells (FIG. 11f).

Treatment of Induced Tumors with Exosomes and CRISPR-Cas9.

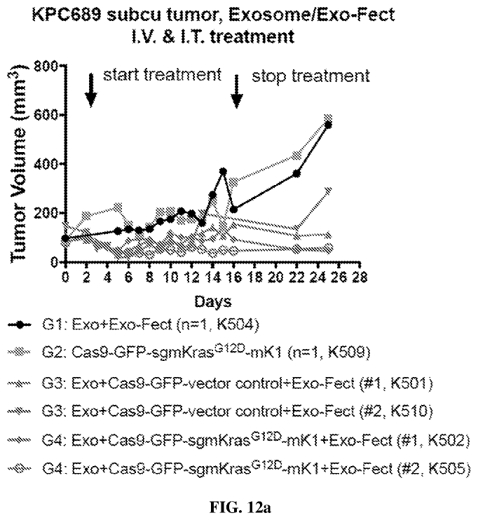

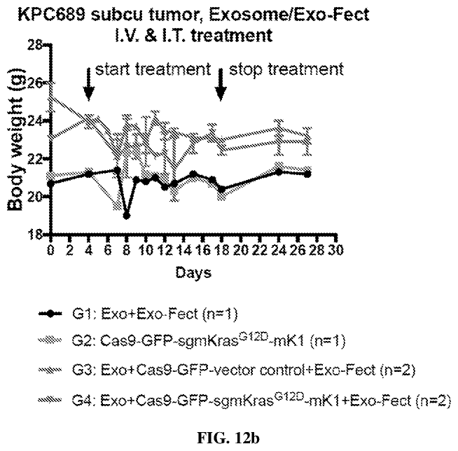

KPC689 cells were implanted subcutaneously into the back of the mice. The mice were divided into four groups, and treated as shown below (FIGS. 12a and 12b). Mice in each group were injected intravenously (I.V.) and intratumorally (I.T.) every day for two weeks and tumor volume was assessed (FIG. 12a). Treatment with exosomes and transfection agent did not slow tumor growth, however treatment with exosomes, the Cas9 with guide RNA plasmid and transfection agent prevented tumor growth over the treatment period and beyond (FIG. 12a). Bodyweight of the mice was also assessed, and treatment in all groups did not negatively affect bodyweight (FIG. 12b).

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,162,282
U.S. Pat. No. 4,310,505
U.S. Pat. No. 4,533,254
U.S. Pat. No. 4,728,575
U.S. Pat. No. 4,728,578
U.S. Pat. No. 4,737,323
U.S. Pat. No. 4,870,287
U.S. Pat. No. 4,897,355
U.S. Pat. No. 4,921,706
U.S. Pat. No. 4,946,787
U.S. Pat. No. 5,049,386
U.S. Pat. No. 5,739,169
U.S. Pat. No. 5,760,395
U.S. Pat. No. 5,801,005
U.S. Pat. No. 5,824,311
U.S. Pat. No. 5,830,880
U.S. Pat. No. 5,846,945
U.S. Pat. No. 5,962,016
U.S. Pat. No. 6,680,068
U.S. Pat. No. 8,030,453
U.S. Patent Appln. Publn 2004/0208921
Almoguera et al., Most human carcinomas of the exocrine pancreas contain mutant c-K-ras genes. Cell, 53:549-554, 1988.
Alvarez-Erviti et al., Delivery of siRNA to the mouse brain by systemic injection of targeted exosomes. Nature Biotechnology, 29:341-345, 2011.
Austin-Ward and Villaseca, Gene therapy and its applications. Rev. Med. Chil., 126:838-845, 1998.
Baietti et al., Syndecan-syntenin-ALIX regulated the biogenesis of exosomes. Nat. Cell Biol., 14:677-685, 2012.
Biankin et al., Pancreatic cancer genomes reveal aberrations in axon guidance pathway genes. Nature, 491:399-405, 2012.
Bukowski et al., Signal transduction abnormalities in T lymphocytes from patients with advanced renal carcinoma: clinical relevance and effects of cytokine therapy. Clin. Cancer Res., 4:2337-2347, 1998.
Chang et al., Pancreatic cancer genomics. Current Opinion in Genetics & Development, 24:74-81, 2014.
Christodoulides et al., Immunization with recombinant class 1 outer-membrane protein from Neisseria meningitidis: influence of liposomes and adjuvants on antibody avidity, recognition of native protein and the induction of a bactericidal immune response against meningococci. Microbiology, 144:3027-3037, 1998.
Clayton et al., Antigen-presenting cell exosomes are protected from complement-mediated lysis by expression of CD55 and CD59. European Journal of Immunology, 33:522-531, 2003.
Collins et al., Oncogenic Kras is required for both the initiation and maintenance of pancreatic cancer in mice. The Journal of Clinical Investigation, 122:639-653, 2012a.
Collins et al., Metastatic pancreatic cancer is dependent on oncogenic Kras in mice. PLoS One, 7:e49707, 2012b.
Combes et al., A new flow cytometry method of platelet-derived microvesicle quantitation in plasma, Thromb. Haemost., 77:220, 1997.
Cooper et al., Systemic exosomal siRNA delivery reduced alpha-synuclein aggregates in brains of transgenic mice. Movement Disorders, 29:1476-1485, 2014.
Davidson et al., Intralesional cytokine therapy in cancer: a pilot study of GM-CSF infusion in mesothelioma. J. Immunother., 21:389-398, 1998.
Du et al., A systematic analysis of the silencing effects of an active siRNA at all single-nucleotide mismatched target sites. Nucleic Acids Research, 33:1671-1677, 2005.
El-Andaloussi et al., Extracellular vesicles: biology and emerging therapeutic opportunities. Nature Reviews Drug Discovery, 12:347-357, 2013.
El-Andaloussi et al., Exosome-mediated delivery of siRNA in vitro and in vivo. Nature Protocols, 7:2112-2126, 2012.
Eser et al., Oncogenic KRAS signalling in pancreatic cancer. British Journal of Cancer, 111:817-822, 2014.
Gomes-da-Silva et al., Lipid-based nanoparticles for siRNA delivery in cancer therapy: paradigms and challenges. Accounts of Chemical Research, 45:1163-1171, 2012.
Gysin et al., Therapeutic strategies for targeting ras proteins. Genes & Cancer, 2:359-372, 2011.
Hanibuchi et al., Therapeutic efficacy of mouse-human chimeric anti-ganglioside GM2 monoclonal antibody against multiple organ micrometastases of human lung cancer in NK cell-depleted SCID mice. Int. J. Cancer, 78:480-485, 1998.
Hellstrand et al., Histamine and cytokine therapy. Acta Oncol., 37:347-353, 1998.
Hingorani et al., Trp53R172H and KrasG12D cooperate to promote chromosomal instability and widely metastatic pancreatic ductal adenocarcinoma in mice. Cancer Cell, 7:469-483, 2005.
Hollander, Immunotherapy for B-cell lymphoma: current status and prospective advances. Front Immunol., 3:3, 2013.
Howlader et al., SEER Cancer Statistics Review, 1975-2011, National Cancer Institute. Bethesda, Md. On the World Wide Web at seercancergov/csr/1975_2011/, 2013.
Hruban et al., K-ras oncogene activation in adenocarcinoma of the human pancreas. A study of 82 carcinomas using a combination of mutant-enriched polymerase chain reaction analysis and allele-specific oligonucleotide hybridization. The American Journal of Pathology, 143:545-554, 1993.
Hui and Hashimoto, Pathways for Potentiation of Immunogenicity during Adjuvant-Assisted Immunizations with Plasmodium falciparum Major Merozoite Surface Protein 1. Infec. Immun., 66:5329-5336, 1998.
Ji et al., Ras activity levels control the development of pancreatic diseases. Gastroenterology, 137:1072-1082, 82 el-6, 2009.
Johnsen et al., A comprehensive overview of exosomes as drug delivery vehicles—endogenous nanocarriers for targeted cancer therapy. Biochimica et Biophysica Acta, 1846:75-87, 2014.
Kahlert et al., Identification of Double Stranded Genomic DNA Spanning all Chromosomes with Mutated KRAS and p53 DNA in the Serum Exosomes of Patients with Pancreatic Cancer. The Journal of biological chemistry 2014.
Kowal et al., Biogenesis and secretion of exosomes. Current Opinion in Cell Biology, 29:116-125, 2014.
Losche et al., Platelet-derived microvesicles transfer tissue factor to monocytes but not to neutrophils, Platelets, 15: 109-115, 2004.
Luga et al., Exosomes mediate stromal mobilization of autocrine Wnt-PCP signaling in breast cancer cell migration. Cell, 151:1542-1556, 2012.
Ma et al., Structural basis for overhang-specific small interfering RNA recognition by the PAZ domain. Nature, 429:318-322, 2004.
Marcus and Leonard, FedExosomes: Engineering Therapeutic Biological Nanoparticles that Truly Deliver. Pharmaceuticals (Basel), 6:659-680, 2013.
Melo et al., Glypican-1 identifies cancer exosomes and detects early pancreatic cancer. Nature, 523:177-182, 2015.
Mesri and Altieri, Endothelial cell activation by leukocyte microparticles, J. Immunol., 161:4382-4387, 1998.
Morel et al., Cellular microparticles: a disseminated storage pool of bioactive vascular effectors, Curr. Opin. Hematol., 11:156-164, 2004.
Ozdemir et al., Depletion of carcinoma-associated fibroblasts and fibrosis induces immunosuppression and accelerates pancreas cancer with reduced survival. Cancer Cell, 25:719-734, 2014.
PCT International Application Publication WO1986/000238.
PCT International Application Publication WO1990/004943.
PCT International Application Publication WO1991/116024.
PCT International Application Publication WO1991/117424.
PCT International Application Publication WO2002/100435.
PCT International Application Publication WO2003/015757.
PCT International Application Publication WO2004/029213.
PCT International Application Publication WO2015/085096.
Pecot et al., Therapeutic Silencing of KRAS using Systemically Delivered siRNAs. Molecular Cancer Therapeutics, 13:2876-2885, 2014.
Peinado et al., Melanoma exosomes educate bone marrow progenitor cells toward a pro-metastatic phenotype through MET. Nature Medicine, 18:883-891, 2012.
Poliseno et al., A coding-independent function of gene and pseudogene mRNAs regulates tumour biology. Nature, 465:1033-1038, 2010.
Qin et al., Interferon-beta gene therapy inhibits tumor formation and causes regression of established tumors in immune-deficient mice. Proc. Natl. Acad. Sci. U.S.A., 95:14411-14416, 1998.
Rachagani et al., Activated KrasG12D is associated with invasion and metastasis of pancreatic cancer cells through inhibition of E-cadherin. Br. J. Cancer, 104:1038-1048, 2011.
Rejiba et al., K-ras oncogene silencing strategy reduces tumor growth and enhances gemcitabine chemotherapy efficacy for pancreatic cancer treatment. Cancer Science, 98:1128-1136, 2007.
Siegel et al., Cancer statistics, 2014. CA: A cancer journal for clinicians, 64:9-29, 2014.
Simoes et al., Cationic liposomes for gene delivery. Expert Opinion on Drug Delivery, 2:237-254, 2005.
Smakman et al., Dual effect of Kras(D12) knockdown on tumorigenesis: increased immune-mediated tumor clearance and abrogation of tumor malignancy. Oncogene, 24:8338-8342, 2005.
Sun et al., Characterization of the mutations of the K-ras, p53, p16, and SMAD4 genes in 15 human pancreatic cancer cell lines. Oncology Reports, 8:89-92, 2001.
Thery et al., Exosomes: composition, biogenesis and function. Nature Reviews Immunology, 2:569-579, 2002.
Valadi et al., Exosome-mediated transfer of mRNAs and microRNAs is a novel mechanism of genetic exchange between cells. Nature Cell Biology, 9:654-659, 2007.
van den Boom et al., Exosomes as nucleic acid nanocarriers. Advanced Drug Delivery Reviews, 65:331-335, 2013.
van der Meel et al., Extracellular vesicles as drug delivery systems: Lessons from the liposome field. Journal of Controlled Release, 195:72-85, 2014.
Wahlgren et al., Plasma exosomes can deliver exogenous short interfering RNA to monocytes and lymphocytes. Nucleic Acids Research, 40:e130, 2012.
Xue et al., Small RNA combination therapy for lung cancer. Proceedings of the National Academy of Sciences USA, 111:E3553-3561, 2014.
Ying et al., Oncogenic Kras maintains pancreatic tumors through regulation of anabolic glucose metabolism. Cell, 149:656-670, 2012.
Yuan et al., Development of siRNA payloads to target KRAS-mutant cancer. Cancer Discovery, 4:1182-1197, 2014.
Zorde Khvalevsky et al., Mutant KRAS is a druggable target for pancreatic cancer. Proceedings of the National Academy of Sciences USA, 110:20723-20728, 2013.

What is claimed is:

1. A composition comprising exosomes, wherein the exosomes comprise CD47 on their surface and wherein the exosomes comprise a CRISPR system.

2. The composition of claim 1, wherein the CRISPR system comprises an endonuclease and a guide RNA (gRNA).

3. The composition of claim 2, wherein the endonuclease is a Cas endonuclease.

4. The composition of claim 3, wherein the endonuclease is a Cas9 endonuclease.

5. The composition of claim 2, wherein the endonuclease is a Cpf1 endonuclease.

6. The composition of claim 2, wherein the guide RNA is a single gRNA.

7. The composition of claim 6, wherein the single gRNA is a CRISPR-RNA (crRNA).

8. The composition of claim 6, wherein the single gRNA comprises a fusion of a crRNA and a trans-activating CRISPR RNA (tracrRNA).

9. The composition of claim 2, wherein the guide RNA comprises a crRNA and a tracrRNA.

10. The composition of claim 2, wherein the endonuclease and the gRNA are encoded on a single nucleic acid molecule within the exosomes.

11. The composition of claim 1, wherein the CRISPR system targets a disease-causing mutation.

12. The composition of claim 11, wherein the disease-causing mutation is a cancer-causing mutation.

13. The composition of claim 12, wherein the cancer-causing mutation is an activating mutation in an oncogene.

14. The composition of claim 12, wherein the cancer-causing mutation is an inhibitory mutation in a tumor suppressor gene.

15. The composition of claim 12, wherein the cancer-causing mutation is Kras^{G12D}.

16. The composition of claim 2, wherein at least 50% of the exosomes comprise an endonuclease and a gRNA.

17. The composition of claim 16, wherein at least 60% of the exosomes comprise an endonuclease and a gRNA.

18. The composition of claim 17, wherein at least 70% of the exosomes comprise an endonuclease and a gRNA.

19. The composition of claim 18, wherein at least 80% of the exosomes comprise an endonuclease and a gRNA.

20. The composition of claim 19, wherein at least 90% of the exosomes comprise an endonuclease and a gRNA.

21. A pharmaceutical composition comprising exosomes of any one of claim 1-20 and an excipient.

22. The composition of claim 21, wherein the composition is formulated for parenteral administration.

23. The composition of claim 22, wherein the composition is formulated for intravenous, intramuscular, sub-cutaneous, or intraperitoneal injection.

24. The composition of claim 22, further comprising an antimicrobial agent.

25. The composition of claim 24, wherein the antimicrobial agent is benzalkonium chloride, benzethonium chloride, benzyl alcohol, bronopol, centrimide, cetylpyridinium chloride, chlorhexidine, chlorobutanol, chlorocresol, chloroxylenol, cresol, ethyl alcohol, glycerin, exetidine, imidurea, phenol, phenoxyethanol, phenylethl alcohol, phenlymercuric nitrate, propylene glycol, or thimerosal.

26. A method of treating a disease in a patient in need thereof comprising administering a composition of any one of claims 21-25 to the patient, thereby treating the disease in the patient.

27. The method of claim 26, wherein administration causes gene editing in the diseased cells in the patient.

28. The method of claim 26, wherein the disease is a cancer.

29. The method of claim 28, wherein the cancer is pancreatic ductal adenocarcinoma.

30. The method of claim 26, wherein the administration is systemic administration.

31. The method of claim 30, wherein the systemic administration is intravenous administration.

32. The method of claim 26, further comprising administering at least a second therapy to the patient.

33. The method of claim 32, wherein the second therapy comprises a surgical therapy, chemotherapy, radiation therapy, cryotherapy, hormonal therapy, or immunotherapy.

34. The method of claim 26, wherein the patient is a human.

35. The method of claim 34, wherein the exosomes are autologous to the patient.

36. A composition comprising exosomes for use in the treatment of a disease in a patient, wherein the exosomes comprise CD47 on their surface and wherein the exosomes comprises a CRISPR system.

37. The composition of claim 36, wherein the CRISPR system comprises an endonuclease and a guide RNA (gRNA).

38. The composition of claim 37, wherein the endonuclease is a Cas endonuclease.

39. The composition of claim 38, wherein the endonuclease is a Cas9 endonuclease.

40. The composition of claim 37, wherein the endonuclease is a Cpf1 endonuclease.

41. The composition of claim 37, wherein the guide RNA is a single gRNA.

42. The composition of claim 41, wherein the single gRNA is a CRISPR-RNA (crRNA).

43. The composition of claim 41, wherein the single gRNA comprises a fusion of a crRNA and a trans-activating CRISPR RNA (tracrRNA).

44. The composition of claim 37, wherein the guide RNA comprises a crRNA and a tracrRNA.

45. The composition of claim 36, wherein the endonuclease and the gRNA are encoded on a single nucleic acid molecule within the exosomes.

46. The composition of claim 36, wherein the CRISPR system targets a disease-causing mutation.

47. The composition of claim 46, wherein the disease-causing mutation is a cancer-causing mutation.

48. The composition of claim 47, wherein the cancer-causing mutation is an activating mutation in an oncogene.

49. The composition of claim 47, wherein the cancer-causing mutation is an inhibitory mutation in a tumor suppressor gene.

50. The composition of claim 47, wherein the cancer-causing mutation is Kras^{G12D}.

51. The composition of claim 37, wherein at least 50% of the exosomes comprise an endonuclease and a gRNA.

52. The composition of claim 51, wherein at least 60% of the exosomes comprise an endonuclease and a gRNA.

53. The composition of claim 52, wherein at least 70% of the exosomes comprise an endonuclease and a gRNA.

54. The composition of claim 53, wherein at least 80% of the exosomes comprise an endonuclease and a gRNA.

55. The composition of claim 54, wherein at least 90% of the exosomes comprise an endonuclease and a gRNA.

56. The composition of claim 36, wherein administration causes gene editing in the diseased cells in the patient.

57. The composition of claim 36, wherein the disease is a cancer.

58. The composition of claim 57, wherein the cancer is pancreatic ductal adenocarcinoma.

59. The composition of claim 36, wherein the composition is formulated for parenteral administration.

60. The composition of claim 59, wherein the composition is formulated for intravenous, intramuscular, sub-cutaneous, or intraperitoneal injection.

61. The composition of claim 59, further comprising an antimicrobial agent.

62. The composition of claim 61, wherein the antimicrobial agent is benzalkonium chloride, benzethonium chloride, benzyl alcohol, bronopol, centrimide, cetylpyridinium chloride, chlorhexidine, chlorobutanol, chlorocresol, chloroxylenol, cresol, ethyl alcohol, glycerin, exetidine, imidurea, phenol, phenoxyethanol, phenylethl alcohol, phenlymercuric nitrate, propylene glycol, or thimerosal.

63. The composition of claim 36, further comprising at least a second therapy.

64. The composition of claim 63, wherein the second therapy comprises a surgical therapy, chemotherapy, radiation therapy, cryotherapy, hormonal therapy, or immunotherapy.

65. The composition of claim 36, wherein the patient is a human.

66. The composition of claim 65, wherein the exosomes are autologous to the patient.

67. Use of exosomes in the manufacture of a medicament for the treatment of a disease, wherein the exosomes comprise CD47 on their surface and wherein the exosomes comprise a CRISPR system.

68. The use of claim 67, wherein the CRISPR system comprises an endonuclease and a guide RNA (gRNA).

69. The use of claim 68, wherein the endonuclease is a Cas endonuclease.

70. The use of claim 69, wherein the endonuclease is a Cas9 endonuclease.

71. The use of claim 68, wherein the endonuclease is a Cpf1 endonuclease.

72. The use of claim 68, wherein the guide RNA is a single gRNA.

73. The use of claim 72, wherein the single gRNA is a CRISPR-RNA (crRNA).

74. The use of claim 72, wherein the single gRNA comprises a fusion of a crRNA and a trans-activating CRISPR RNA (tracrRNA).

75. The use of claim 68, wherein the guide RNA comprises a crRNA and a tracrRNA.

76. The use of claim 68, wherein the endonuclease and the gRNA are encoded on a single nucleic acid molecule within the exosomes.

77. The use of claim 67, wherein the CRISPR system targets a disease-causing mutation.

78. The use of claim 77, wherein the disease-causing mutation is a cancer-causing mutation.

79. The use of claim 78, wherein the cancer-causing mutation is an activating mutation in an oncogene.

80. The use of claim 78, wherein the cancer-causing mutation is an inhibitory mutation in a tumor suppressor gene.

81. The use of claim 78, wherein the cancer-causing mutation is Kras^{G12D}.

82. The use of claim 68, wherein at least 50% of the exosomes comprise an endonuclease and a gRNA.

83. The use of claim 82, wherein at least 60% of the exosomes comprise an endonuclease and a gRNA.

84. The use of claim 83, wherein at least 70% of the exosomes comprise an endonuclease and a gRNA.

85. The use of claim 84, wherein at least 80% of the exosomes comprise an endonuclease and a gRNA.

86. The use of claim 85, wherein at least 90% of the exosomes comprise an endonuclease and a gRNA.

87. The use of claim 67, wherein the disease is a cancer.

88. The use of claim 87, wherein the cancer is pancreatic ductal adenocarcinoma.

89. The use of claim 67, wherein the medicament is formulated for parenteral administration.

90. The use of claim 67, wherein the medicament is formulated for systemic administration.

91. The use of claim 89, wherein the medicament is formulated for intravenous, intramuscular, sub-cutaneous, or intraperitoneal injection.

92. The use of claim 67, wherein the medicament comprises an antimicrobial agent.

93. The use of claim 92, wherein the antimicrobial agent is benzalkonium chloride, benzethonium chloride, benzyl alcohol, bronopol, centrimide, cetylpyridinium chloride, chlorhexidine, chlorobutanol, chlorocresol, chloroxylenol, cresol, ethyl alcohol, glycerin, exetidine, imidurea, phenol, phenoxyethanol, phenylethl alcohol, phenlymercuric nitrate, propylene glycol, or thimerosal.
